SEQUENCE LISTING (21 sequences)

SEQ ID NO: 1 - protein, 40 aa, Homo Sapiens
DAEFRHDSGYEVHHQKLVFFAEDVGSNKGAIIGLMVGGVV

SEQ ID NO: 2 - protein, 769 aa, Homo Sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 3 - DNA, 645 bp, Mus Musculus
gaggtgaagcttctcgagtctggaggtggcctggtgcagcctggaggatccctgaaactctcctgtgcagcctcaggattcgattttagtagatactggatgaattgggtccggcaggctccagggaaagggctagaatggattggagaaattaatccagatagcagtacgataaactatacgccatctctaaaggataaattcatcatctccagagacaacgccaaaaatacgctgtacctgcaaatgagcaaagtgagatctgaggacacagccctttattactgtgcaagacaaatgggctactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgt

SEQ ID NO: 4 - DNA, 642 bp, Mus Musculus
gatatccagatgacacagactacatcctccctgtctgcctctctgggagacagagtcaccatcagttgcagtgcaagtcagggcattagcaattatttaaactggtttcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtataggaagcttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 5 - protein, 215 aa, Mus Musculus
EVKLLESGGGLVQPGGSLKLSCAASGFDFSRYWMNWVRQAPGKGLEWIGEINPDSSTINYTPSLKDKFIISRDNAKNTLYLQMSKVRSEDTALYYCARQMGYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDC

SEQ ID NO: 6 - protein, 214 aa, Mus Musculus
DIQMTQTTSSLSASLGDRVTISCSASQGISNYLNWFQQKPDGTVKLLIYYTSSLHSGVPSRFSGSGSGTDYSLTISNLEPEDIATYYCQQYRKLPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 7 - DNA, 684 bp, Mus Musculus
gttactctgaaagagtctggccctgggatattgaagccctcacagaccctcagtctgacttgttctttctctgggttttcactgagcacttctggtatgggtgtaggctggattcgtcagtcttcagggaagggtctggagtggctggcacacatttggtgggatgatgataagtactataacccatccctgaagagccagctcacaatctccaaggatacctccagaaaccaggtattcctcaagatcaccagtgtggacactgcagatactgccacttactactgtgctcgaaggggggtacgacatagagactactttgactactggggccaaggcaccactctcacagtctcctcagccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccc

SEQ ID NO: 8 - protein, 228 aa, Mus Musculus
VTLKESGPGILKPSQTLSLTCSFSGFSLSTSGMGVGWIRQSSGKGLEWLAHIWWDDDKYYNPSLKSQLTISKDTSRNQVFLKITSVDTADTATYYCARRGVRHRDYFDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINP

SEQ ID NO: 9 - DNA, 657 bp, Mus Musculus
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaaactccttatctacaaagtttccaaccgattttctggggtcccagacaggttcagtggcagtggatcagggacagatttcacactcaagatcagcagagtggaggctgaggatctgggagtttattactgctttcaaggttcacgtgttcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 10 - protein, 219 aa, Mus Musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSRVPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - protein, 42 aa, Homo Sapiens [sequence omitted]

SEQ ID NO: 12 - protein, 43 aa, Homo Sapiens
DAEFRHDSGYEVHHQKLVFFAEDVGSNKGAIIGLMVGGVVIAT

SEQ ID NO: 13 - protein, 41 aa, Homo Sapiens
DAEFRHDSGYEVHHQKLVFFAEDVGSNKGAIIGLMVGGVVI

SEQ ID NO: 14 - protein, 40 aa, Artificial Sequence
  Synthetic Construct
DAEFRHDSGYEVHHQKLVFFAEDVGSNKGAIIGLMAGGVV

SEQ ID NO: 15 - protein, 40 aa, Artificial Sequence
  Synthetic Construct
DAEFRHDSGYEVHHQKLVFFAEDVGSNKGAIIGLMVAGVV

SEQ ID NO: 16 - protein, 40 aa, Artificial Sequence
  Synthetic Construct
DAEFRHDSGYEVHHQKLVFFAEDVGSNKGAIIGLMVGAVV

SEQ ID NO: 17 - protein, 40 aa, Artificial Sequence
  Synthetic Construct
DAEFRHDSGYEVHHQKLVFFAEDVGSNKGAIIGLMVGGVA

SEQ ID NO: 18 - protein, 40 aa, Artificial Sequence; identical to SEQ ID NO: 17
  Synthetic Construct

SEQ ID NO: 19 - protein, 108 aa, Artificial Sequence
  Synthetic Construct
DIQMTQSPSSLSASVGDRVTITCSASQGISNYLNWYQQKPGKAPKLLIYYTSSLHSGVPSRFSGSGSGTDFTFTISSLQPEDIATYYCQQYRKLPYTFGGGTKVEIKR

SEQ ID NO: 20 - protein, 107 aa, Artificial Sequence
  Synthetic Construct
EVQLVESGGGLVQPGGSLRLSCAASYWMNWVRQAPGKGLEWVSEINPDSSTINYTPSLKDRFTISRDNAKNTLYLQMNSLRAEDTAVYYCARQMGYWGQGTTLTVSS

SEQ ID NO: 21 - protein, 107 aa, Artificial Sequence
  Synthetic Construct
QVQLQESGPGLVKPSETLSLTCTVSYWMNWIRQPPGKGLEWIGEINPDSSTINYTPSLKDRVTISKDTSKNQFSLKLSSVTAADTAVYYCARQMGYWGQGTLVTVSS

(19) United States
(12) Patent Application Publication
(10) Pub. No.: US 2004/0146512 A1
(43) Pub. Date: Jul. 29, 2004

(54) Methods of treating Alzheimer's disease using antibodies directed against amyloid beta peptide and compositions thereof

(76) Inventors: Arnon Rosenthal, Woodside, CA (US); Gil Levkowitz, Tel Aviv (IL)

Correspondence Address: MORRISON & FOERSTER LLP, 755 PAGE MILL RD, PALO ALTO, CA, 94304-1018, US

(21) Appl. No.: 10/683,815

(22) Filed: Oct. 9, 2003

Related U.S. Application Data
(60) Provisional application No. 60/417,232, filed on Oct. 9, 2002.
(60) Provisional application No. 60/447,611, filed on Feb. 13, 2003.
(60) Provisional application No. 60/464,754, filed on Apr. 22, 2003.
(60) Provisional application No. 60/480,353, filed on Jun. 20, 2003.

(51) Int. Cl. A61K 39/395

(52) U.S. Cl. 424/146.1

(57) ABSTRACT

Monoclonal antibodies directed against amyloid beta peptide and methods of using same for treatment and prevention of Alzheimer's disease and Down's syndrome are described.

### CROSS-REFERENCES TO RELATED APPLICATIONS

This application claims the benefit of U.S. provisional application Ser. Nos. 60/417,232, filed Oct. 9, 2002; 60/447,611, filed Feb. 13, 2003; 60/464,754, filed Apr. 22, 2003; and 60/480,353, filed Jun. 20, 2003, which are incorporated in their entirety by reference.

### TECHNICAL FIELD

The present invention relates generally to detection and treatment of disease associated with expression of amyloid-beta peptide (Aβ), such as Alzheimer's disease and Down's syndrome. The invention is more specifically related to antibodies directed against Aβ and its precursor, βAPP. The invention thus provides immunotherapeutic compositions and methods useful in the detection and treatment of disease associated with over-expression (or accumulation) of Aβ and βAPP.

### BACKGROUND OF THE INVENTION

Alzheimer's disease (AD) is a degenerative brain disorder characterized clinically by progressive memory deficits, confusion, gradual physical deterioration and, ultimately, death. Approximately 15 million people worldwide are affected by Alzheimer's disease, and the number is expected to increase dramatically as lifespans increase. Histologically, the disease is characterized by neuritic plaques, found primarily in the association cortex, limbic system and basal ganglia. The major constituent of these plaques is amyloid beta peptide (Aβ), which is the cleavage product of beta amyloid precursor protein (βAPP or APP). APP is a type I transmembrane glycoprotein that contains a large ectopic N-terminal domain, a transmembrane domain, and a small cytoplasmic C-terminal tail. Alternative splicing of the transcript of the single APP gene on chromosome 21 results in several isoforms that differ in the number of amino acids.

Aβ appears to have a central role in the neuropathology of Alzheimer's disease. Familial forms of the disease have been linked to mutations in APP and the presenilin genes (Tanzi et al., 1996, Neurobiol. Dis. 3:159-168; Hardy, 1996, Ann. Med. 28:255-258). Diseased-linked mutations in these genes result in increased production of the 42-amino acid form of Aβ, the predominant form found in amyloid plaques. Moreover, immunization of transgenic mice that overexpress a disease-linked mutant form of APP with human Aβ reduces plaque burden and associated pathologies (Schenk et al., 1999, Nature 400:173-177), and peripheral administration of antibodies directed against Aβ also reduces plaque burden in the brain (Bard et al., 2000, Nature Medicine 6(8):916-919).

Antibody therapy therefore provides a promising approach to the treatment and prevention of Alzheimer's disease. There remains a need for antibodies and other immunotherapeutic agents directed against Aβ having improved efficacy, and which are suitable for use with human patients.

Throughout this application various publications (including patents and patent applications) are referenced. The disclosures of these publications in their entireties are hereby incorporated by reference.

### SUMMARY OF THE INVENTION

The invention provides isolated monoclonal antibodies that bind to Aβ peptide (SEQ ID NO:1) (Table 6). More specifically, antibodies are provided that bind to amino acids 1-16, 16-28 or 28-40 of Aβ peptide. Preferably, the antibodies competitively inhibit binding of a monoclonal antibody having the amino acid sequence shown in SEQ ID NO: 4, 6, 8 or 10 (Tables 9 and 11), or the binding of the monoclonal antibody produced by the hybridoma designated 8A1.2A1, 3C6.1F9 or 10B10.2E6. In some embodiments, the monoclonal antibody binds the Aβ peptide with an affinity of about 200 nM or less, about 100 nM or less, about 60 nM or less, preferably about 30 nM or less, more preferably, about 3 nM or less, about 2 nM or less, and about 1 nM or less. In some embodiments, the Fab fragments of the monoclonal antibody binds the Aβ peptide with an affinity of about 200 nM or less, about 100 nM or less, about 60 nM or less, about 30 nM or less, about 3 nM or less, about 2 nM or less, and about 1 nM or less. In preferred embodiments, the antibody binds the same Aβ epitope to which a monoclonal antibody having the amino acid sequence shown in SEQ ID NO: 4, 6, 8 or 10, or the monoclonal antibody produced by the hybridoma designated 8A1.2A1, 3C6.1F9, or 10B10.2E6 binds. The invention also provides a monoclonal antibody produced by the hybridoma designated 8A1.2A1, 3C6.1F9, or 10B10.2E6. The monoclonal antibody described herein can optionally be conjugated to a therapeutic agent and/or labeled with a detectable marker.

In another aspect, the invention provides isolated antibodies that preferentially bind to amino acids 28-40 of Aβ peptide (SEQ ID NO:1) (Table 6). In some embodiments, the antibodies are monoclonal antibodies. In some embodiments, the antibody preferentially binds to an epitope that includes amino acid 39 and/or 40 of the Aβ peptide (SEQ ID NO:1). In some embodiments, the antibodies bind to an epitope that includes amino acid 39 and/or 40 of the Aβ peptide (SEQ ID NO: 1) but show no significant cross-reactivity with Aβ₁₋₄₂ and/or Aβ₁₋₄₃ peptide. In some embodiments, the antibodies bind to an epitope that includes amino acids 36-40 of the Aβ peptide (SEQ ID NO:1). In some embodiments, the antibodies bind to an epitope that includes amino acids 36-40 of the Aβ peptide (SEQ ID NO: 1), but show no significant cross-reactivity with Aβ₁₋₄₂ and Aβ₁₋₄₃ peptide. In some embodiments, the antibody binds to the Aβ peptide (SEQ ID NO:1) with an affinity of about 200 nM or less, about 100 nM or less, about 60 nM or less, about 30 nM or less, about 3 nM or less, about 2 nM or less, about 1 nM or less. In some embodiments, the Fab fragment of the antibody binds to the Aβ peptide (SEQ ID NO:1) with an affinity of about 200 nM or less, about 100 nM or less, about 60 nM or less, about 30 nM or less, about 3 nM or less, about 2 nM or less, about 1 nM or less. In some embodiments, the Fab fragment of the antibody that preferentially binds to an epitope that includes amino acid 39 and/or 40 of the Aβ peptide (SEQ ID NO: 1) binds to the Aβ peptide (SEQ ID NO: 1) with an affinity of about 200 nM or less, about 100 nM or less, about 60 nM or less, about 30 nM or less, about 3 nM or less, about 2 nM or less, about 1 nM or less. In some embodiments, the Fab fragment of the antibody that preferentially binds to an epitope that includes amino acids 36-40 of the Aβ peptide (SEQ ID NO: 1) binds to the Aβ peptide (SEQ ID NO:1) with an affinity of about 200 nM or less, about 100 nM or less, about 60 nM or less, about 30 nM or less, about 3 nM or less, about 2 nM or less, about 1 nM or less. In some embodiments, the antibody competitively inhibits binding of a monoclonal antibody comprising the amino acid sequences shown in SEQ ID NO:4 and/or 6, or the monoclonal antibody produced by the hybridoma designated 8A1.2A1 to Aβ₁₋₄₀ peptide (SEQ ID NO:1). In some embodiments, the antibody binds to the epitope that a monoclonal antibody comprising the amino acid sequences shown in SEQ ID NO:4 and/or 6, or the monoclonal antibody produced by the hybridoma designated 8A1.2A1 binds. In some embodiments, the antibody is a human antibody. In some embodiments, the antibody comprises the amino acid sequences shown in SEQ ID NO:4 and 6, or is produced by the hybridoma designated 8A1.2A1.

In another aspect, the invention is a humanized antibody derived from a monoclonal antibody comprising the amino acid sequences shown in SEQ ID NO:4 and/or 6, or the monoclonal antibody produced by the hybridoma designated 8A1.2A1. In some embodiments, the humanized antibody comprises one or more CDRs of the monoclonal antibody comprising the amino acid sequences shown in SEQ ID NO:4 and/or 6, or the monoclonal antibody produced by the hybridoma designated 8A1.2A1. In another aspect, the invention provides a humanized antibody that binds to the same epitope(s) as the monoclonal antibody comprising the amino acid sequences shown in SEQ ID NO:4 and/or 6, or the monoclonal antibody produced by the hybridoma designated 8A1.2A1. Generally, a humanized antibody of the invention comprises one or more (one, two, three, four, five, six) CDRs which are the same and/or derived from the CDR(s) of the monoclonal antibody comprising the amino acid sequences shown in SEQ ID NO:4 and/or 6, or the monoclonal antibody produced by the hybridoma designated 8A1.2A1.

In anther aspect, the invention is a chimeric antibody comprising variable regions derived from variable regions of a heavy chain and a light chain of monoclonal antibody comprising the amino acid sequences shown in SEQ ID NO:4 and/or 6, or the monoclonal antibody produced by the hybridoma designated 8A1.2A1, and constant regions derived from constant regions of a heavy chain and a light chain of a human antibody.

In some embodiments, the binding affinity of the antibodies disclosed herein is about 100 pM or less, about 50 pM or less, about 25 pM or less, about 15 pM or less, about 10 pM or less, about 5 pM or less, or about 2 pM or less.

In addition, the invention provides an isolated monoclonal antibody that binds to βAPP (SEQ ID NO:2) (Table 7) and that competitively inhibits binding of the monoclonal antibody produced by the hybridoma designated 25E12.1F9.1H8 (BP26), 24H4.2E10.1F5 (BP27), 1F10.8E6.2A2 (BP80), 13E12.1C5 (BP81), or 14D9.1G8 (BP82).

Compositions comprising one or more antibodies of the invention are also provided. In some embodiments, the composition comprises at least two antibodies, a first antibody directed against amino acids 16-28 of Aβ peptide and a second antibody directed against amino acids 28-40 of Aβ peptide. Optionally, the composition further comprises a physiologically acceptable carrier.

The invention further provides an isolated polynucleotide that encodes a monoclonal antibody as described herein, as well as a vector comprising the polynucleotide and a host cell containing the vector. Such expression systems can be used in a method of producing an immunoreactive polypeptide, such as an antibody of the invention, wherein the host cell is cultured and the polypeptide produced by the cultured host cell is recovered. In some embodiments, the polynucleotide or the vector of the invention comprises nucleotide sequence shown in SEQ ID NO:3 and/or 5 (Table 8). In some embodiments, the polynucleotide or the vector of the invention comprises nucleotide sequence shown in SEQ ID NO:7 and/or 9 (Table 10). The invention also provides a host cell comprising a vector described herein. In another aspect, the invention also provides a method of producing an immunoreactive polypeptide comprising culturing the host cell described herein and recovering the polypeptide so produced. The invention also provides an immunoreactive polypeptide produced by culturing the host cell described herein and recovering the polypeptide so produced.

The invention also provides a pharmaceutical composition comprising an effective amount of any of the polypeptides (including any of the antibodies) or polynucleotides described herein and a pharmaceutical acceptable carrier. In some embodiments, the pharmaceutical composition comprises an antibody that preferentially binds to amino acids 16-28 of Aβ peptide. In some embodiments, the pharmaceutical composition comprises an antibody that preferentially binds to amino acids 28-40 of Aβ peptide (SEQ ID NO: 1). In some embodiments, the antibody is a monoclonal antibody. In some embodiments, the pharmaceutical composition comprises an antibody that preferentially binds to an epitope that includes amino acid 39 and/or 40 of the Aβ peptide (SEQ ID NO:1). In some embodiments, the antibodies that bind to an epitope that includes amino acid 39 and/or 40 of the Aβ peptide (SEQ ID NO: 1) but show no significant cross-reactivity with Aβ1-42 and/or Aβ₁₋₄₃ peptide. In some embodiments, the antibodies that bind to an epitope that includes amino acids 36-40 of the Aβ peptide (SEQ ID NO:1) but show no significant cross-reactivity with Aβ₁₋₄₂ and/or Aβ₁₋₄₃ peptide. In some embodiments, the antibody that binds to amino acids 28-40 of Aβ peptide (SEQ ID NO: 1) binds to the Aβ peptide (SEQ ID NO:1) with an affinity of about 200 nM or less, about 100 nM or less, about 60 nM or less, about 30 nM or less, about 3 nM or less, about 2 nM or less, about 1 nM or less. In some embodiments, the Fab fragment of the antibody binds to the Aβ peptide (SEQ ID NO: 1) with an affinity of about 200 nM or less, about 100 nM or less, about 60 nM or less, about 30 nM or less, about 3 nM or less, about 2 nM or less, about 1 nM or less. In some embodiments, the Fab fragment of the antibody that preferentially binds to an epitope that includes amino acid 39 and/or 40 of the Aβ peptide (SEQ ID NO:1) binds to the Aβ peptide (SEQ ID NO:1) with an affinity of about 200 nM or less, about 100 nM or less, about 60 nM or less, about 30 nM or less, about 3 nM or less, about 2 nM or less, about 1 nM or less. In some embodiments, the Fab fragment of the antibody that preferentially binds to an epitope that includes amino acids 36-40 of the Aβ peptide (SEQ ID NO: 1) binds to the Aβ peptide (SEQ ID NO: 1) with an affinity of about 200 nM or less, about 100 nM or less, about 60 nM or less, about 30 nM or less, about 3 nM or less, about 2 nM or less, about 1 nM or less. In some embodiments, the antibody competitively inhibits binding of a monoclonal antibody comprising the amino acid sequences shown in SEQ ID NO:4 and/or 6, or the monoclonal antibody produced by the hybridoma designated 8A1.2A1. In some embodiments, the antibody binds to the epitope on Aβ peptide (SEQ ID NO: 1) that an antibody comprising amino acid sequence shown in SEQ ID NO:4 and/or 6, or the monoclonal antibody produced by the hybridoma designated 8A1.2A1 binds. In other embodiments, the pharmaceutical composition comprises a human antibody, a humanized or a chimeric antibody derived from any of the antibodies described herein.

The invention also provides a hybridoma designated 8A1.2A1, 3C6.1F9, or 10B10.2E6.

The invention also provides a method for preventing, treating, inhibiting, or delaying the development of Alzheimer's disease and other diseases associated with altered Aβ or βAPP expression, or accumulation of Aβ peptide, such as Down's syndrome, Parkinson's disease, multi-infarct dementia. The method comprises administering an effective dosage a pharmaceutical composition comprising an antibody (including polypeptides) or polynucleotide of the invention to a subject. In some embodiments, the pharmaceutical composition comprises an antibody that preferentially binds to amino acids 16-28 of Aβ peptide. In some embodiments, the antibody binds preferentially to amino acids 28-40 of Aβ peptide (SEQ ID NO:1). In some embodiments, the antibody preferentially binds to an epitope that includes amino acid 39 and/or 40 of the Aβ peptide (SEQ ID NO: 1). In some embodiments, the antibody preferentially binds to an epitope that includes amino acids 36-40 of the Aβ peptide (SEQ ID NO: 1). In some embodiments, the antibodies that bind to an epitope that includes amino acid 39 and/or 40 of the Aβ peptide (SEQ ID NO: 1) but show no significant cross-reactivity with Aβ₁₋₄₂ and/or Aβ₁₋₄₃ peptide. In some embodiments, the antibodies that bind to an epitope that includes amino acids 36-40 of the Aβ peptide (SEQ ID NO:1) but show no significant cross-reactivity with Aβ₁₋₄₂ and/or Aβ₁₋₄₃ peptide. In some embodiments, the antibody that binds to amino acids 28-40 of Aβ peptide (SEQ ID NO: 1) binds to the Aβ peptide (SEQ ID NO: 1) with an affinity of about 200 nM or less, about 100 nM or less, about 60 nM or less, about 30 nM or less, about 3 nM or less, about 2 nM or less, about 1 nM or less. In some embodiments, the Fab fragment of the antibody binds to the Aβ peptide (SEQ ID NO: 1) with an affinity of about 200 nM or less, about 100 nM or less, about 60 nM or less, about 30 nM or less, about 3 nM or less, about 2 nM or less, about 1 nM or less. In some embodiments, the Fab fragment of the antibody that preferentially binds to an epitope that includes amino acid 39 and/or 40 of the Aβ peptide (SEQ ID NO: 1) binds to the Aβ peptide (SEQ ID NO: 1) with an affinity of about 200 nM or less, about 100 nM or less, about 60 nM or less, about 30 nM or less, about 3 nM or less, about 2 nM or less, about 1 nM or less. In some embodiments, the Fab fragment of the antibody that preferentially binds to an epitope that includes amino acids 36-40 of the Aβ peptide (SEQ ID NO: 1) binds to the Aβ peptide (SEQ ID NO:1) with an affinity of about 200 nM or less, about 100 nM or less, about 60 nM or less, about 30 nM or less, about 3 nM or less, about 2 nM or less, about 1 nM or less. In some embodiments, the antibody competitively inhibits binding of a monoclonal antibody comprising the amino acid sequences shown in SEQ ID NO:4 and/or 6, or the monoclonal antibody produced by the hybridoma designated 8A1.2A1. The antibody can be a chimeric, human or humanized antibody, and can be a fragment of an antibody. Examples of antibody fragments include, but are not limited to, Fab, F(ab′)₂, and Fv fragments. The antibody can also be a single chain, bispecific or multispecific antibody that can comprise one or more antibody fragments. The antibody can further be linked to a therapeutic agent, or co-administered with a therapeutic agent.

The invention also provides a method of delaying development of a symptom associated with Alzheimer's disease or other diseases related to accumulation of Aβ peptide in a subject comprising administering an effective dosage of a pharmaceutical composition comprising an antibody (including polypeptides) or polynucleotide of the invention to the subject.

The invention also provides a method of suppressing formation of amyloid plaques in a subject comprising administering an effective dosage of a pharmaceutical composition comprising an antibody (including polypeptides) or polynucleotide of the invention to the subject. In some embodiments, the amyloid plaques are in the brain of the subject.

The invention also provides a method of reducing amyloid plaques in a subject comprising administering an effective dosage of a pharmaceutical composition comprising an antibody (including polypeptides) or polynucleotide of the invention to the subject. In some embodiments, the amyloid plaques are in the brain of the subject.

The invention also provides a method of removing or clearing amyloid plaques in a subject comprising administering an effective dosage of a pharmaceutical composition comprising an antibody (including polypeptides) or polynucleotide of the invention to the subject. In some embodiments, the amyloid plaques are in the brain of the subject.

Additionally, the invention provides a method for inhibiting the accumulation of Aβ peptide in a tissue comprising contacting the tissue with a monoclonal antibody of the invention.

The invention also provides a method of reducing Aβ peptide (including soluble and deposited form) in the brain of an individual comprising administrating to the individual an effective amount of an antibody of the invention. In some embodiments, the accumulation of Aβ peptide is inhibited and/or reduced in the brain. In some embodiments, the toxic effects of Aβ peptide are inhibited and/or reduced. Thus, the method of the invention can be used to treat any disease in which accumulation of Aβ peptide is present or suspected, such as Alzheimer's disease, Down's syndrome, Parkinson's disease, and multi-infarct dementia.

In some embodiments of the methods described herein, the composition is administered by systemic injection. In some embodiments, the composition is administered by intraperitoneal injection.

The composition that can be administered in the methods of the invention described above also includes a composition comprising an antibody that binds to an epitope that includes amino acid 42 and/or 43 of Aβ₁₋₄₃ (SEQ ID NO:12), an antibody that binds to C-terminal end of Aβ₁₋₄₃ (SEQ ID NO:12) but show no significant cross-reactivity with Aβ₁₋₄₂ (SEQ ID NO: 11) and/or Aβ₁₋₄₀ (SEQ ID NO:1), an antibody that binds to an epitope that includes amino acid 41 and/or 42 of Aβ₁₋₄₂ (SEQ ID NO:11), or an antibody that binds to C-terminal end of Aβ₁₋₄₂ (SEQ ID NO:11) but show no significant cross-reactivity with Aβ₁₋₄₃ (SEQ ID NO:12) and/or Aβ₁₋₄₀ (SEQ ID NO:1).

Antibodies of the invention can further be used in the detection, diagnosis and monitoring of Alzheimer's disease and other diseases associated with altered Aβ or βAPP expression, such as Down's syndrome. The method comprises contacting a specimen of a patient suspected of having altered Aβ or βAPP expression with an antibody of the invention and determining whether the level of Aβ or βAPP differs from that of a control or comparison specimen.

In a further embodiment, the invention provides articles of manufacture and kits containing materials useful for treating pathological conditions such as Alzheimer's disease or other disease associated with altered Aβ or βAPP expression or detecting or purifying Aβ or βAPP. The article of manufacture comprises a container with a label. Suitable containers include, for example, bottles, vials, and test tubes. The containers may be formed from a variety of materials such as glass or plastic. The container holds a composition having an active agent which is effective for treating pathological conditions or for detecting or purifying Aβ or βAPP. The active agent in the composition is an antibody and preferably, comprises monoclonal antibodies specific for Aβ or βAPP or any other composition of the invention. The label on the container indicates that the composition is used for treating pathological conditions such as Alzheimer's disease or detecting or purifying Aβ or βAPP, and may also indicate directions for either in vivo or in vitro use, such as those described above.

The kit of the invention comprises the container described above and a second container comprising a buffer. It may further include other materials desirable from a commercial and user standpoint, including other buffers, diluents, filters, needles, syringes, and package inserts with instructions for use in any of the methods described herein.

The invention also provides any of the compositions described (such as the antibodies) for any of the use described herein whether in the context of use as a medicament and/or use for manufacture of a medicament.

### BRIEF DESCRIPTION OF THE FIGURES

FIG. 1 is a bar graph demonstrating that the monoclonal antibodies directed against Aβ do not cross-react with βAPP.

FIG. 2 is a bar graph demonstrating that all of the monoclonal antibodies directed against Aβ capture soluble Aβ.

FIG. 3 is a bar graph demonstrating that antibodies produced by the hybridoma 8A1.2A1 preferentially bind to an epitope includes amino acid 39 and/or 40 of Aβ₁₋₄₀.

FIG. 4 shows quantification of total A-beta, thioflavine-S, and MHC-II staining in frontal cortex and hippocampus following intracranial injection of antibody 2286, 2324, 2289, or a control antibody anti-amnesiac.

FIG. 5 shows anti-Aβ2286 F_{(ab′)2} fragments do not activate microglia, nor do they remove compact amyloid deposits as effectively as the complete anti-Aβ 2286 IgG. Panels A-D show CD45 immunohistochemistry in the hippocampus. Panels E-H show total Aβ immunohistochemistry in the hippocampus. Panels I-L show thioflavine-S staining in the hippocampus. Mice were injected with intact anti-Aβ2286 IgG (A, E and I), anti-Aβ 2286 F_{(ab′)2} fragments (B, F and J), control (anti-amnesiac) IgG (C, G and K), or control (anti-amnesiac) F_{(ab′)2} fragments (D, H and L). Magnification=40X. Scale bar=120 μm.

FIG. 6 shows quantification of CD45 and total Aβ immunohistochemistry and thioflavine-S staining following intracranial injection of anti-Aβ 2286 antibodies and anti-Aβ 2286 F_{(ab′)2} fragments. Panel A shows the ratio of right to left sides for CD45 immunohistochemistry. Panel B shows the ratio of right to left sides for total Aβ immunohistochemistry. Panel C shows the ratio of right to left sides for thioflavine-S staining. The solid bars indicate values for frontal cortex, and the open bars indicate values for hippocampus. On the x-axis, IgG-Cont refers to control (anti-amnesiac) intact IgG; F(ab′)2-Cont refers to control (anti-amnesiac) F_{(ab′)2} fragments; IgG-Abeta refers to anti-Aβ intact IgG; F(ab′)2-Abeta refers to anti-Aβ F_{(ab′)2} fragments. “***” indicates P<0.001, and “*” indicates P<0.05 as compared to both control antibody groups. Lines over bars indicate P values for comparisons between the specific pair of groups indicated.

FIG. 7 shows Aβ serum levels (top graph) and anti-Aβ antibody concentrations in the serum (bottom graph) following systemic injection of antibody 2286. Each point in the graph represents Aβ serum level or anti-Aβ antibody concentration of one mouse treated under the condition as indicated. The line in the graph represents average Aβ serum level or anti-Aβ antibody concentration of mice treated under the condition as indicated.

FIG. 8 shows binding of antibody 2286 and antibody 2324 to different Aβ peptide variants.

### DETAILED DESCRIPTION OF THE INVENTION

The present invention provides monoclonal antibodies that are specific for Aβ peptide and for βAPP. The anti-Aβ antibodies disclosed herein bind with high affinity and without cross-reactivity with βAPP, making them particularly suitable for use in methods for detecting and treating Alzheimer's disease and other diseases associated with altered Aβ expression, such as Down's syndrome. In one embodiment, the invention provides antibodies directed against C terminal portions of Aβ peptide. In one embodiment, the C terminal portion of Aβ peptide to which the antibody is directed includes amino acids 28-40. In some embodiments, the antibody preferentially binds to an epitope that includes amino acid 39 and/or 40 of Aβ₁₋₄₀. In some embodiments, the antibody preferentially binds to an epitope that includes amino acids 36-40 of Aβ₁₋₄₀. In some embodiments, the antibody binds to the C terminal portion Aβ₁₋₄₀ with an affinity of about 200 nM or less, about 150 nM or less, about 100 nM or less, about 60 nM or less, about 30 nM or less, or about 3 nM or less, about 2 nM or less, about 1 nM or less. In some embodiments, antibody preferentially binds to an epitope that includes amino acid 39 and/or 40 of Aβ₁₋₄₀ with an affinity of about 200 nM or less, about 150 nM or less, about 100 nM or less, about 60 nM or less, about 30 nM or less, or about 3 nM or less, about 2 nM or less, about 1 nM or less. In some embodiments, antibody preferentially binds to an epitope that includes amino acids 36-40 of Aβ₁₋₄₀ with an affinity of about 200 nM or less, about 150 nM or less, about 100 nM or less, about 60 nM or less, about 30 nM or less, or about 3 nM or less, about 2 nM or less, about 1 nM or less. The therapeutic utility of antibodies directed against the C terminal portion of Aβ peptide is based on the surprising discovery that such antibodies are capable of removing Aβ deposits and thioflavine-S deposits (indicative of a toxic fibrillar form of deposits) in brain tissue of an animal model for Alzheimer's disease.

The discovery that antibodies directed to amino acids 28-40 (in some embodiments, epitope that includes amino acid 39 and/or 40 of Aβ (SEQ ID NO: 1), or amino acids 36-40 of Aβ (SEQ ID NO: 1)) of Aβ are effective at removing fibrillar deposits in an animal model of Alzheimer's disease contrasts with the reports of others. As reviewed by Schenk (October 2002, Nat. Rev. Neurosci. 3(10):824-8), not all anti-Aβ antibodies can effectively reduce pathology in the brain, and those that can reduce pathology are limited to antibodies directed against the first 16 amino acids of Aβ (Bacskai et al., 2002, J. Neurosci. 22(18):7873-8; Bard et al., 2000, Nature Med. 6:916-919), or amino acids 16-28 (DeMattos et al., 2001, Proc. Nat'l Acad. Sci. 98(15):8850-55; DeMattos et al., 2002, Science 295(5563):2264-7; Dodart et al., 2002 Nat. Neuroscience 5(5):452-7). In contrast, antibodies directed to the carboxy terminal (e.g., amino acids 33-42) failed to reduce amyloid burden in the brain (Bacskai 2002, supra; Bard 2000, supra).

Definitions

All scientific and technical terms used in this application have meanings commonly used in the art unless otherwise specified. As used in this application, the following words or phrases have the meanings specified.

As used herein, “antibody” includes intact immunoglobulin or antibody molecules, polyclonal antibodies, multispecific antibodies (e.g., bispecific antibodies formed from at least two intact antibodies) and immunoglobulin fragments (such as Fab, F(ab′)₂, or Fv), so long as they exhibit any of the desired specific binding properties described herein. Antibodies are typically proteins or polypeptides that exhibit binding specificity to a specific antigen.

As used herein, “monoclonal antibody” refers to an antibody obtained from a population of substantially homogeneous antibodies, ie., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example.

As used herein, “humanized” antibodies refers to forms of non-human (e.g. murine) antibodies that are specific chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Preferred are antibodies having Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody.

The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Chothia et al. (1989) Nature 342:877). As used herein, a CDR may refer to CDRs defined by either approach or by a combination of both approaches.

As used herein, “human antibody” means an antibody having an amino acid sequence corresponding to that of an antibody produced by a human and/or has been made using any of the techniques for making human antibodies known in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, PNAS, (USA) 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boemer et al., 1991, J. Immunol., 147 (1):86-95; and U.S. Pat. No. 5,750,373.

“Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. Typically, in these chimeric antibodies, the variable region of both light and heavy chains mimics the variable regions of antibodies derived from one species of mammals, while the constant portions are homologous to the sequences in antibodies derived from another. One clear advantage to such chimeric forms is that, for example, the variable regions can conveniently be derived from presently known sources using readily available hybridomas or B cells from non human host organisms in combination with constant regions derived from, for example, human cell preparations. While the variable region has the advantage of ease of preparation, and the specificity is not affected by its source, the constant region being human, is less likely to elicit an immune response from a human subject when the antibodies are injected than would the constant region from a non-human source. However, the definition is not limited to this particular example.

A “functional Fc region” possesses at least one effector function of a native sequence Fc region. Exemplary “effector functions” include Clq binding; complement dependent cytotoxicity (CDC); Fc receptor binding; antibody-dependent cell-mediated cytotoxicity (ADCC); phagocytosis; down-regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95% sequence identity therewith.

As used herein “antibody-dependent cell-mediated cytotoxicity” and “ADCC” refer to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, e.g., in a animal model such as that disclosed in Clynes et al., 1998, PNAS (USA), 95:652-656.

As used herein, “human effector cells” means leukocytes that express one or more FcRs and perform effector functions. Preferably, the cells express at least FcγRIII and perform ADCC effector function. Examples of human leukocytes that mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g. from blood or PBMCs.

As used herein, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

“Complement dependent cytotoxicity” and “CDC” refer to the lysing of a target in the presence of complement. The complement activation pathway is initiated by the binding of the first component of the complement system (Clq) to a molecule (e.g. an antibody) complexed with a cognate antigen. To assess complement activation, a CDC assay, e.g. as described in Gazzano-Santoro et al., J. Immunol. Methods, 202:163 (1996), may be performed.

As used herein, “affinity matured” antibody means an antibody with one or more alterations in one or more CDRs thereof that result an improvement in the affinity of the antibody for antigen compared to a parent antibody that does not possess the alteration(s). Preferred affinity matured antibodies will have nanomolar or even picomolar affinities for the target antigen. Affinity matured antibodies are produced by procedures known in the art (Marks et al., 1992, Bio/Technology, 10:779-783; Barbas et al., 1994, Proc Nat. Acad. Sci, USA 91:3809-3813; Schier et al., 1995, Gene, 169:147-155; Yelton et al., 1995, J. Immunol., 155:1994-2004; Jackson et al., 1995, J. Immunol., 154(7):3310-9; Hawkins et al, 1992, J. Mol. Biol., 226:889-896).

As used herein, “immunospecific” binding of antibodies refers to the antigen specific binding interaction that occurs between the antigen-combining site of an antibody and the specific antigen recognized by that antibody (i.e., the antibody reacts with the protein in an ELISA or other immunoassay, and does not react detectably with unrelated proteins).

An epitope that “specifically binds”, or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a Aβ₁₋₄₀ epitope is an antibody that binds this epitope with greater affinity, avidity, more readily, and/or with greater duration than it binds to other Aβ₁₋₄₀ epitopes or non-Aβ₁₋₄₀ epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “polypeptide” includes proteins, fragments of proteins, and peptides, whether isolated from natural sources, produced by recombinant techniques or chemically synthesized. Polypeptides of the invention typically comprise at least about 6 amino acids.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

As used herein, “adjuvant” includes those adjuvants commonly used in the art to facilitate an immune response. Examples of adjuvants include, but are not limited to, helper peptide; aluminum salts such as aluminum hydroxide gel (alum) or aluminum phosphate; Freund's Incomplete Adjuvant and Complete Adjuvant (Difco Laboratories, Detroit, Mich.); Merck Adjuvant 65 (Merck and Company, Inc., Rahway, N.J.); AS-2 (Smith-Kline Beecham); QS-21 (Aquilla Biopharmaceuticals); MPL or 3d-MPL (Corixa Corporation, Hamilton, Mont.); LEIF; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A; muramyl tripeptide phosphatidyl ethanolamine or an immunostimulating complex, including cytokines (e.g., GM-CSF or interleukin-2, -7 or -12) and immunostimulatory DNA sequences. In some embodiments, such as with the use of a polynucleotide vaccine, an adjuvant such as a helper peptide or cytokine can be provided via a polynucleotide encoding the adjuvant.

As used herein, an “effective dosage” or “effective amount” drug, compound, or pharmaceutical composition is an amount sufficient to effect beneficial or desired results. For prophylactic use, beneficial or desired results includes results such as eliminating or reducing the risk, lessening the severity, or delaying the outset of the disease, including biochemical, histologic and/or behavioral symptoms of the disease, its complications and intermediate pathological phenotypes presenting during development of the disease. For therapeutic use, beneficial or desired results includes clinical results such as inhibiting or suppressing the formation of amyloid plaques, reducing, removing, or clearing amyloid plaques, improving cognition or reversing cognitive decline, sequestering soluble Aβ peptide circulating in biological fluids, decreasing one or more symptoms resulting from the disease (biochemical, histologic and/or behavioral), including its complications and intermediate pathological phenotypes presenting during development of the disease, increasing the quality of life of those suffering from the disease, decreasing the dose of other medications required to treat the disease, enhancing effect of another medication, delaying the progression of the disease, and/or prolonging survival of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, “treatment” or “treating” is an approach for obtaining beneficial or desired results including clinical results. For purposes of this invention, beneficial or desired clinical results include, but are not limited to, one or more of the following: inhibiting or suppressing the formation of amyloid plaques, reducing, removing, or clearing amyloid plaques, improving cognition or reversing cognitive decline, sequestering soluble Aβ peptide circulating in biological fluids, reducing Aβ peptide (including soluble and deposited) in a tissue (such as brain), inhibiting and/or reducing accumulation of Aβ peptide in the brain, inhibiting and/or reducing toxic effects of Aβ peptide in a tissue (such as brain), decreasing symptoms resulting from the disease, increasing the quality of life of those suffering from the disease, decreasing the dose of other medications required to treat the disease, delaying the progression of the disease, and/or prolonging survival of patients.

As used herein, “delaying” development of Alzheimer's disease means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of Alzheimer's disease is a method that reduces probability of disease development in a given time frame and/or reduces extent of the disease in a given time frame, when compared to not using the method. Such comparisons are typically based on clinical studies, using a statistically significant number of subjects.

“Development” of Alzheimer's disease means the onset and/or progression of Alzheimer's disease within an individual. Alzheimer's disease development can be detectable using standard clinical techniques as described herein. However, development also refers to disease progression that may be initially undetectable. For purposes of this invention, progression refers to the biological course of the disease state, in this case, as determined by a standard neurological examination, or patient interview or may be determined by more specialized testing. A variety of these diagnostic tests include, but not limited to, neuroimaging, detecting alterations of levels of specific proteins in the serum or cerebrospinal fluid (e.g., amyloid peptides and Tau), computerized tomography (CT), and magnetic resonance imaging (MRI). “Development” includes occurrence, recurrence, and onset. As used herein “onset” or “occurrence” of Alzheimer's disease includes initial onset and and/or recurrence.

As used herein, a “at risk” individual is an individual who is at risk of development of Alzheimer's disease. An individual “at risk” may or may not have detectable disease, and may or may not have displayed detectable disease prior to the treatment methods described herein. “At risk” denotes that an individual has one or more so-called risk factors, which are measurable parameters that correlate with development of Alzheimer's disease. An individual having one or more of these risk factors has a higher probability of developing Alzheimer's disease than an individual without these risk factor(s). These risk factors include, but are not limited to, age, sex, race, diet, history of previous disease, presence of precursor disease, genetic (i.e., hereditary) considerations, and environmental exposure.

As used herein, “a” or “an” means at least one, unless clearly indicated otherwise.

Antibodies

The invention provides isolated monoclonal antibodies (including human, humanized or chimeric antibodies of the invention) that bind to Aβ peptide (SEQ ID NO:1). More specifically, antibodies are provided that bind to amino acids 1-16, 16-28 or 28-40 of Aβ peptide. In some embodiment, the antibodies preferentially binds to an epitope that includes amino acid 39 and/or 40 of the Aβ peptide (SEQ ID NO: 1). An antibody that binds to Aβ peptide containing amino acids 1-40 of SEQ ID NO: 1, but does not bind (as is understood by one skilled in the art, does not significantly bind) to Aβ peptide containing amino acids 1-38 of SEQ ID NO:1, is an antibody that preferentially binds to an epitope that includes amino acid 39 and/or 40 of the Aβ peptide (SEQ ID NO:1). In some embodiments, the antibody binds to an epitope that includes amino acids 36-40 of Aβ peptide (SEQ ID NO: 1). In some embodiments, the antibody binds to amino acids 28-40 of Aβ peptide (SEQ ID NO:1) with an affinity of about 200 nM or less, about 60 nM or less, about 30 nM or less, about 3 nM or less, or about 1 nM or less. In some embodiments, the antibody preferentially binds to an epitope that includes amino acid 39 and/or 40 of the Aβ peptide (SEQ ID NO: 1) with an affinity of about 60 nM or less, about 30 nM or less, or about 3 nM or less. In some embodiments, the antibody preferentially binds to an epitope that includes amino acid 39 and/or 40 of the Aβ peptide (SEQ ID NO: 1) with an affinity of about 3 nM or less. Preferably, the antibodies competitively inhibit binding of a monoclonal antibody having the amino acid sequence shown in SEQ ID NO: 4, 6, 8, and/or 10, or the binding of the monoclonal antibody produced by the hybridoma designated 8A1.2A1, 3C6.1F9 or 10B10.2E6. In some embodiments, the monoclonal antibody binds the Aβ peptide with an affinity of about 60 nM or less, preferably about 30 nM or less, and more preferably, about 3 nM or less. In preferred embodiments, the antibody binds the same Aβ epitope to which a monoclonal antibody having the amino acid sequence shown in SEQ ID NO: 4, 6, 8, and/or 10, or the monoclonal antibody produced by the hybridoma designated 8A1.2A1, 3C6.1F9, or 10B10.2E6 binds. The monoclonal antibody can optionally be conjugated to a therapeutic agent and/or labeled with a detectable marker.

In some embodiments and as described herein (and is known in the art), affinity is measured using the corresponding Fab fragment of the antibody.

In addition, the invention provides an isolated monoclonal antibody that binds to βAPP (SEQ ID NO:2) and that competitively inhibits binding of the monoclonal antibody produced by the hybridoma designated 25E12.1F9.1H8 (BP26), 24H4.2E10.1F5 (BP27), 1F10.8E6.2A2 (BP80), 13E12.1C5 (BP81), or 14D9.1G8 (BP82).

In another aspect, the invention provides a humanized antibody derived from a monoclonal antibody having the amino acid sequence shown in SEQ ID NO: 4 and/or 6, or the monoclonal antibody produced by the hybridoma designated 8A1.2A1. A humanized form of the antibody may or may not have CDRs identical to the monoclonal antibody derived from. Determination of CDR regions is well within the skill of the art. In some embodiments, the invention provides an antibody which comprises at least one CDR that is substantially homologous to at least one CDR, at least two, at least three, at least four, at least 5 CDRs of the monoclonal antibody (or, in some embodiments substantially homologous to all 6 CDRs of the monoclonal antibody, or derived from the monoclonal antibody) derived from. Other embodiments include antibodies which have at least two, three, four, five, or six CDR(s) that are substantially homologous to at least two, three, four, five or six CDRs of the monoclonal antibody or derived from the monoclonal antibody. It is understood that, for purposes of this invention, binding specificity and/or overall activity (which may be in terms of clearing Aβ deposit) is generally retained, although the extent of activity may vary compared to the monoclonal antibody produced by the hybridoma designated 8A1.2A1. The invention also provides methods of making any of these antibodies. Methods of making antibodies are known in the art and are described herein.

Competition assays can be used to determine whether two antibodies bind the same epitope by recognizing identical or sterically overlapping epitopes. Typically, antigen is immobilized on a multi-well plate and the ability of unlabeled antibodies to block the binding of labeled antibodies is measured. Common labels for such competition assays are radioactive labels or enzyme labels.

One way of determining binding affinity of antibodies to Aβ peptide is by measuring affinity of monofunctional Fab fragments of the antibodies. To obtain monofunction Fab fragments, antibodies, for example, IgGs can be cleaved with papain or expressed recombinantly. Affinities of anti-Aβ Fab fragments of monoclonal antibodies can be determined by Surface Plasmon Resonance (SPR) system (BIAcore ₃₀₀₀™, BIAcore, Inc., Piscaway, N.J.). SA chips (streptavidin) are used according to the supplier's instructions. Biotinylated Aβ peptide 1-40 (SEQ ID NO: 1) can be diluted into HBS-EP (100 mM HEPES pH 7.4, 150 mM NaCl, 3 mM EDTA, 0.005% P20) and injected over the chip at a concentration of 0.005 mg/mL. Using variable flow time across the individual chip channels, two ranges of antigen density are achieved: 10-20 response units (RU) for detailed kinetic studies and 500-600 RU for concentration. Regeneration studies showed that a mixture of Pierce elution buffer and 4 M NaCl (2:1) effectively removed the bound Fab while keeping the activity of Aβ peptide on the chip for over 200 injections. HBS-EP buffer can be used as running buffer for all the BIAcore assays. Serial dilutions (0.1-10× estimated KD) of purified Fab samples are injected for 2 min at 100 μL/min and dissociation times of up to 30 h min are allowed. The concentrations of the Fab proteins can be determined by ELISA and/or SDS-PAGE electrophoresis using a standard Fab of known concentration (determined by amino acid analysis). Kinetic association rates (kon) and dissociation rates (koff) are obtained simultaneously by fitting the data to a 1:1 Langmuir binding model (Lofas & Johnsson, 1990) using the BIAevaluation program. Equilibrium dissociation constant (KD) values are calculated as koff/kon.

The invention provides antibodies in monomeric, dimeric and multivalent forms. For example, bispecific antibodies, monoclonal antibodies that have binding specificities for at least two different antigens, can be prepared using the antibodies disclosed herein. Methods for making bispecific antibodies are known in the art (see, e.g., Suresh et al., 1986, Methods in Enzymology 121:210). Traditionally, the recombinant production of bispecific antibodies was based on the coexpression of two immunoglobulin heavy chain-light chain pairs, with the two heavy chains having different specificities (Millstein and Cuello, 1983, Nature 305, 537-539).

According to one approach to making bispecific antibodies, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690, published Mar. 3, 1994.

Heteroconjugate antibodies, comprising two covalently joined antibodies, are also within the scope of the invention. Such antibodies have been used to target immune system cells to unwanted cells (U.S. Pat. No. 4,676,980), and for treatment of HIV infection (PCT application publication Nos. WO 91/00360 and WO 92/200373; EP 03089). Heteroconjugate antibodies may be made using any convenient cross-linking methods. Suitable cross-linking agents and techniques are well known in the art, and are described in U.S. Pat. No. 4,676,980.

In certain embodiments, the immunoreactive molecule is an antibody fragment. Various techniques have been developed for the production of antibody fragments. These fragments can be derived via proteolytic digestion of intact antibodies (see, e.g., Morimoto et al., 1992, J. Biochem. Biophys. Methods 24:107-117 and Brennan et al., 1985, Science 229:81), or produced directly by recombinant host cells. For example, Fab′-SH fragments can be directly recovered from E. coli and chemically coupled to form F(ab′)₂ fragments (Carter et al., 1992, Bio/Technology 10:163-167). In another embodiment, the F(ab′)₂ is formed using the leucine zipper GCN4 to promote assembly of the F(ab′)₂ molecule. According to another approach, Fv, Fab or F(ab′)₂ fragments are isolated directly from recombinant host cell culture.

The monoclonal antibody of the invention can be provided in the form of a pharmaceutical composition, optionally together with a carrier.

The antibody also may be entrapped in microcapsules prepared, for example, by coacervation techniques or by interfacial polymerization (for example, hydroxymethylcellulose or gelatin-microcapsules and poly(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington 's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000. To increase the serum half life of the antibody, one may incorporate a salvage receptor binding epitope into the antibody (especially an antibody fragment) as described in U.S. Pat. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

The antibodies disclosed herein may also be formulated as immunoliposomes. Liposomes containing the antibody are prepared by methods known in the art, such as described in Epstein et al., 1985, Proc. Natl. Acad. Sci. USA 82:3688; Hwang et al., 1980, Proc. Natl Acad. Sci. USA 77:4030; and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. In addition, Fab′ fragments of the antibody of the present invention can be conjugated to the liposomes as described in Martin et al., 1982, J. Biol. Chem. 257:286-288, via a disulfide interchange reaction.

In some embodiments, the antibodies of the invention are single chain (ScFv), mutants thereof, fusion proteins comprising an antibody portion, humanized antibodies, chimeric antibodies, diabodies linear antibodies, single chain antibodies, and any other modified configuration of the immunoglobulin molecule.

Production of Antibodies

Monoclonal antibodies may be prepared using hybridoma methods, such as those described by Kohler and Milstein, 1975, Nature 256:495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods, such as those described in U.S. Pat. No. 4,816,567. DNA encoding the monoclonal antibodies is isolated and sequenced using conventional procedures, such as by using oligonucleotide probes that are capable of binding specifically to genes encoding the heavy and light chains of the monoclonal antibodies. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells.

The DNA can be modified, for example, by covalently joining to the immunoglobulin coding sequence all or part of the coding sequence for a non-immunoglobulin polypeptide. In that manner, “chimeric” or “hybrid” antibodies are prepared that have the binding specificity of a monoclonal antibody disclosed herein. Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for one surface epitope of Aβ (or βAPP) and another antigen-combining site having specificity for a different antigen.

The invention also encompasses humanized antibodies. Therapeutic antibodies often elicit adverse effects, in part due to triggering of an immune response directed against the administered antibody. This can result in reduced drug efficacy, depletion of cells bearing the target antigen, and an undesirable inflammatory response. To circumvent the above, recombinant anti-Aβ humanized antibodies are generated. The polynucleotide sequence of an antibody, such as SEQ ID NO:3 and/or 5 may be used for genetic manipulation to generate a “humanized” antibody, or to improve the affinity, or other characteristics of the antibody. The general principle in humanizing an antibody involves retaining the basic sequence of the antigen-binding portion of the antibody, while swapping the non-human remainder of the antibody with human antibody sequences. There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. For example, the constant region may be engineered to more resemble human constant regions to avoid immune response if the antibody is used in clinical trials and treatments in humans. See, for example, U.S. Pat. Nos. 5,997,867 and 5,866,692.

In the recombinant humanized antibodies, the Fcγ portion can be modified to avoid interaction with Fcγ receptor and the complement immune system. This type of modification was designed by Dr. Mike Clark from the Department of Pathology at Cambridge University, and techniques for preparation of such antibodies are described in WO 99/58572, published Nov. 18, 1999.

A number of “humanized” antibody molecules comprising an antigen-binding site derived from a non-human immunoglobulin have been described, including chimeric antibodies having rodent V regions and their associated complementarity determining regions (CDRs) fused to human constant domains. See, for example, Winter et al. Nature 349:293-299 (1991); Lobuglio et al. Proc. Nat. Acad. Sci. USA 86:4220-4224 (1989); Shaw et al. J Immunol. 138:4534-4538 (1987); and Brown et al. Cancer Res. 47:3577-3583 (1987). Other references describe rodent CDRs grafted into a human supporting framework region (FR) prior to fusion with an appropriate human antibody constant domain. See, for example, Riechmann et al. Nature 332:323-327 (1988); Verhoeyen et al. Science 239:1534-1536 (1988); and Jones et al. Nature 321:522-525 (1986). Another reference describes rodent CDRs supported by recombinantly veneered rodent framework regions. See, for example, European Pat. Publication No. 519,596. These “humanized” molecules are designed to minimize unwanted immunological response toward rodent antihuman antibody molecules which limits the duration and effectiveness of therapeutic applications of those moieties in human recipients. Other methods of humanizing antibodies that may also be utilized are disclosed by Daugherty et al., Nucl. Acids Res., 19:2471-2476 (1991) and in U.S. Pat. Nos. 6,180,377; 6,054,297; 5,997,867; 5,866,692; 6,210,671; 6,350,861; and PCT WO 01/27160.

In yet another alternative, fully human antibodies may be obtained by using commercially available mice which have been engineered to express specific human immunoglobulin proteins. Transgenic animals which are designed to produce a more desirable (e.g., fully human antibodies) or more robust immune response may also be used for generation of humanized or human antibodies. Examples of such technology are Xenomouse™ from Abgenix, Inc. (Fremont, Calif.) and HuMAb-Mouse® and TC Mouse™ from Medarex, Inc. (Princeton, N.J.).

In another alternative, antibodies may be made recombinantly by phage display technology. See, for example, U.S. Pat. Nos. 5,565,332; 5,580,717; 5,733,743 and 6,265,150; and Winter et al., Annu. Rev. Immunol. 12:433-455 (1994). Alternatively, the phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson, Kevin S. and Chiswell, David J., Current Opinion in Structural Biology 3, 564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Mark et al., J. Mol. Biol. 222:581-597 (1991), or Griffith et al., EMBO J. 12:725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” Marks, et al., Bio/Technol. 10:779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266 (1993). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT patent application PCT WO 9306213, published Apr. 1, 1993). Unlike traditional humanization of rodent antibodies by CDR grafting, this technique provides completely human antibodies, which have no framework or CDR residues of rodent origin. It is apparent that although the above discussion pertains to humanized antibodies, the general principles discussed are applicable to customizing antibodies for use, for example, in dogs, cats, primates, equines and bovines.

Chimeric or hybrid antibodies also may be prepared in vitro using known methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Single chain Fv fragments may also be produced, such as described in Iliades et al., 1997, FEBS Letters, 409:437-441. Coupling of such single chain fragments using various linkers is described in Kortt et al., 1997, Protein Engineering, 10:423-433. A variety of techniques for the recombinant production and manipulation of antibodies are well known in the art.

Variant and Modified Immunoreactive Polypeptides and Antibodies

The invention also provides polypeptides comprising an amino acid sequence of the antibodies of the invention, such as a monoclonal antibody having the amino acid sequence shown in SEQ ID NO: 4 and/or 6, or the monoclonal antibody produced by the hybridoma designated 8A1.2A1. Immunoreactive polypeptides described herein are useful for and can be used in any of the compositions, kits, and methods described herein. The polypeptides have one or more of the binding properties described herein, and in some embodiments, display any one or more additional functional properties described herein. In some embodiments, the polypeptide comprises one or more of the light chain and/or heavy chain variable regions of the monoclonal antibody. In some embodiments, the polypeptide comprises one or more (one, two, three, four, five, or six) of the light chain and/or heavy chain CDRs of the monoclonal antibody. In some embodiments, the polypeptide comprises three CDRs of the light chain and/or heavy chain of the monoclonal antibody. In some embodiments, the polypeptide comprises an amino acid sequence of the monoclonal antibody that has any of the following: at least 5 contiguous amino acids of a sequence of the monoclonal antibody, at least 8 contiguous amino acids, at least about 10 contiguous amino acids, at least about 15 contiguous amino acids, at least about 20 contiguous amino acids, at least about 25 contiguous amino acids, at least about 30 contiguous amino acids, wherein at least 3 of the amino acids are from a variable region of the monoclonal antibody. In one embodiment, the variable region is from a light chain of the monoclonal antibody. In another embodiment, the variable region is from a heavy chain of the monoclonal antibody. In another embodiment, the 5 (or more) contiguous amino acids are from a complementarity determining region (CDR) of the monoclonal antibody.

A polypeptide “variant,” as used herein, is a polypeptide that differs from a native protein in one or more substitutions, deletions, additions and/or insertions, such that the immunoreactivity of the polypeptide is not substantially diminished. In other words, the ability of a variant to specifically bind antigen may be enhanced or unchanged, relative to the native protein, or may be diminished by less than 50%, and preferably less than 20%, relative to the native protein. Polypeptide variants preferably exhibit at least about 80%, more preferably at least about 90% and most preferably at least about 95% identity (determined as described herein) to the identified polypeptides.

Amino acid sequence variants of the antibodies are prepared by introducing appropriate nucleotide changes into the antibody DNA, or by peptide synthesis. Such variants include, for example, deletions from, and/or insertions into and/or substitutions of, residues within the amino acid sequences of SEQ ID NO: 4, 6 or 8 described herein. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are preferred locations for mutagenesis is called “alanine scanning mutagenesis,” and is described by Cunningham and Wells, 1989, Science, 244:1081-1085. A residue or group of target residues is identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the serum half-life of the antibody.

Substitution variants have at least one amino acid residue in the antibody molecule removed and a different residue inserted in its place. The sites of greatest interest for substitutional mutagenesis include the hypervariable regions, but FR alterations are also contemplated. Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened.

TABLE 1
Conservative Substitutions

| Original | Preferred | Exemplary |
|---|---|---|
| Residue | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) Hydrophobic: Norleucine, Met, Ala, Val, Leu, Ile;

(2) Neutral hydrophilic: Cys, Ser, Thr;

(3) Acidic: Asp, Glu;

(4) Basic: Asn, Gln, His, Lys, Arg;

(5) Residues that influence chain orientation: Gly, Pro; and

(6) Aromatic: Trp, Tyr, Phe.

Non-conservative substitutions are made by exchanging a member of one of these classes for another class.

Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

A particularly preferred type of substitutional variant involves substituting one or more hypervariable region residues of a parent antibody. Generally, the resulting variant(s) selected for further development will have improved biological properties relative to the parent antibody from which it is generated.

Most preferred are antibodies that have been modified as described in WO 99/58572, published Nov. 18, 1999. These antibodies comprise, in addition to a binding domain directed at the target molecule, an effector domain having an amino acid sequence substantially homologous to all or part of a constant domain of a human immunoglobulin heavy chain. These antibodies are capable of binding the target molecule without triggering significant complement dependent lysis, or cell-mediated destruction of the target. Preferably, the effector domain is capable of specifically binding FcRn and/or FcγRIIb. These are typically based on chimeric domains derived from two or more human immunoglobulin heavy chain C_{H}2 domains. Antibodies modified in this manner are preferred for use in chronic antibody therapy, to avoid inflammatory and other adverse reactions to conventional antibody therapy.

Glycosylation variants of antibodies are variants in which the glycosylation pattern of an antibody is altered. “Altering” means deleting one or mote carbohydrate moieties found in the antibody, adding one or more carbohydrate moieties to the antibody, changing the composition of glycosylation (glycosylation pattern), the extent of glycosylation, etc. Glycosylation variants may, for example, be prepared by removing, changing and/or adding one or more glycosylation sites in the nucleic acid sequence encoding the antibody.

Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Hefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Mature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the antibody can be prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

The glycosylation pattern of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, variations in the glycosylation pattern of the antibodies can be expected (see, e.g. Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5.278,299). Glycosylation, or certain types of glycosylation, can be enzymatically removed from the glycoprotein, for example using endoglycosidase H (Endo H). In addition, the recombinant host cell can be genetically engineered to be defective in processing certain types of polysaccharides. These and similar techniques are well known in the art.

Polypeptides may comprise a signal (or leader) sequence at the N-terminal end of the protein that co-translationally or post-translationally directs transfer of the protein. The polypeptide may also be conjugated to a linker or other sequence for ease of synthesis, purification or identification of the polypeptide (e.g., poly-FEs), or to enhance binding of the polypeptide to a solid support. For example, a polypeptide may be conjugated to an immunoglobulin Fc region.

Portions and other variants having fewer than about 100 amino acids, and generally fewer than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

Polypeptides can be synthesized on a Perkin Elmer/Applied Biosystems Division 430A peptide synthesizer using FMOC chemistry with HPTU (O-BenzotriazoleN,N,N′,N′-tetramethyluronium hexafluorophosphate) activation. A Gly—Cys—Gly sequence may be attached to the amino terminus of the peptide to provide a method of conjugation, binding to an immobilized surface, or labeling of the peptide. Cleavage of the peptides from the solid support may be carried out using the following cleavage mixture: trifluoroacetic acid:ethanedithiol:thioanisole:water:phenol (40:1:2:2:3). After cleaving for 2 hours, the peptides may be precipitated in cold methyl-t-butyl-ether. The peptide pellets may then be dissolved in water containing 0.1% trifluoroacetic acid (TFA) and lyophilized prior to purification by C18 reverse phase HPLC. A gradient of 0%-60% acetonitrile (containing 0.1% TFA) in water may be used to elute the peptides. Following lyophilization of the pure fractions, the peptides may be characterized using electrospray or other types of mass spectrometry and by amino acid analysis.

Fusion Proteins

In some embodiments, the polypeptide is a fusion protein that comprises multiple polypeptides as described herein, or that comprises at least one polypeptide as described herein and an unrelated sequence. Additional fusion partners can be added.

A fusion partner may, for example, serve as an immunological fusion partner by assisting in the provision of T helper epitopes, preferably T helper epitopes recognized by humans. As another example, a fusion partner may serve as an expression enhancer, assisting in expressing the protein at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and the second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located 5′ to the DNA sequence encoding the first polypeptide. Similarly, stop codons required to end translation and transcription termination signals are present 3′ to the DNA sequence encoding the second or final polypeptide.

Fusion proteins are also provided that comprise a polypeptide of the present invention together with an unrelated immunogenic protein. Preferably the immunogenic protein is capable of eliciting a memory response. Examples of such proteins include tetanus, tuberculosis and hepatitis proteins (see, for example, Stoute et al., 1997, New Engl. J. Med. 336:86-91).

Within preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of antigen to antigen presenting cells. Other fusion partners include the non-structural protein from influenzae virus, NS I (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAR This property has been exploited for the development of E. coli CLYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion protein. A repeat portion is found in the C-terminal region starting at residue 178. A particularly preferred repeat portion incorporates residues 188-305.

In general, polypeptides (including fusion proteins) and polynucleotides as described herein are isolated. An “isolated” polypeptide or polynucleotide is one that is removed from its original environment. For example, a naturally occurring protein is isolated if it is separated from some or all of the coexisting materials in the natural system.

Preferably, such polypeptides are at least about 90% pure, more preferably at least about 95% pure and most preferably at least about 99% pure. A polynucleotide is considered to be isolated if, for example, it is cloned into a vector that is not a part of the natural environment.

Polynucleotides and Vectors

The invention further provides isolated polynucleotides that encode a monoclonal antibody as described herein, as well as vectors comprising the polynucleotide and a host cell containing the vector. Such expression systems can be used in a method of producing an immunoreactive polypeptide, such as an antibody of the invention, wherein the host cell is cultured and the polypeptide produced by the cultured host cell is recovered. Polynucleotides encoding antibodies of the invention can also be delivered to a host subject for expression of the antibody by cells of the host subject. Examples of strategies for polynucleotide delivery to and expression of anti-senilin antibodies in the central nervous system of a host subject are described in PCT application No. WO98/44955, published Oct. 15, 1998.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an antibody or a portion thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably at least about 80% identity and most preferably at least about 90% identity to a polynucleotide sequence that encodes a native antibody or a portion thereof

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles ate endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Polynucleotides may be prepared using any of a variety of techniques known in the art. DNA encoding an antibody may be obtained from a cDNA library prepared from tissue expressing antibody mRNA. The antibody-encoding gene may also be obtained from a genomic library or by oligonucleotide synthesis. Libraries can be screened with probes (such as binding partners or oligonucleotides of at least about 20-80 bases) designed to identify the gene of interest or the protein encoded by it. Illustrative libraries include human liver cDNA library (human liver 5′ stretch plus cDNA, Clontech Laboratories, Inc.) and mouse kidney cDNA library (mouse kidney 5′-stretch cDNA, Clontech laboratories, Inc.). Screening the cDNA or genomic library with the selected probe may be conducted using standard procedures, such as those described in Sambrook et al., Molecular Cloning: A Laboratory Manual, New York: Cold Spring Harbor Laboratory Press, 1989. Alternatively, one can isolate the gene encoding antibody using PCR methodology (Sambrook et al., supra; Dieffenbach et al., PCR Primer: A Laboratory Manual, Cold Spring Harbor Laboratory Press, 1995).

The oligonucleotide sequences selected as probes should be sufficiently long and sufficiently unambiguous that false positives are minimized. The oligonucleotide is preferably labeled such that it can be detected upon hybridization to DNA in the library being screened. Methods of labeling are well known in the art, and include the use of radiolabels, such as ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined through sequence alignment using computer software programs, which employ various algorithms to measure homology.

Nucleic acid molecules having protein coding sequence may be obtained by screening selected cDNA or genomic libraries, and, if necessary, using conventional primer extension procedures as described in Sambrook et al., supra, to detect precursors and processing intermediates of mRNA that may not have been reverse-transcribed into cDNA.

Polynucleotide variants may generally be prepared by any method known in the art, including chemical synthesis by, for example, solid phase phosphoramidite chemical synthesis. Modifications in a polynucleotide sequence may also be introduced using standard mutagenesis techniques, such as oligonucleotide-directed site-specific mutagenesis (see Adelman et al., DNA 2:183, 1983). Alternatively, RNA molecules may be generated by in vitro or in vivo transcription of DNA sequences encoding an antibody, or portion thereof, provided that the DNA is incorporated into a vector with a suitable RNA polymerase promoter (such as T7 or SP6). Certain portions may be used to prepare an encoded polypeptide, as described herein. In addition, or alternatively, a portion may be administered to a patient such that the encoded polypeptide is generated in vivo (e.g., by transfecting antigen-presenting cells, such as dendritic cells, with a cDNA construct encoding the polypeptide, and administering the transfected cells to the patient).

Any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl-, methyl-, thio- and other modified forms of adenine, cytidine, guanine, thymine and uridine.

Nucleotide sequences can be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include expression vectors, replication vectors, probe generation vectors and sequencing vectors. In general, a vector will contain an origin of replication functional in at least one organism, convenient restriction endonuclease sites and one or more selectable markers. Other elements will depend upon the desired use, and will be apparent to those of ordinary skill in the art.

Within certain embodiments, polynucleotides may be formulated so as to permit entry into a cell of a mammal, and to permit expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, or vaccinia or other pox virus (e.g., avian pox virus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

Other formulations for therapeutic purposes include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Pharmaceutical Compositions

The invention provides antibodies, polypeptides, and/or polynucleotides that are incorporated into pharmaceutical compositions. In some embodiments, the pharmaceutical composition comprises an antibody that preferentially binds to amino acids 28-40 of Aβ peptide (SEQ ID NO:1). In some embodiments, the antibody is a monoclonal antibody. In some embodiments, the pharmaceutical composition comprises a monoclonal antibody that preferentially binds to an epitope that includes amino acid 39 and/or 40 of the Aβ peptide (SEQ ID NO:1). In some embodiments, the pharmaceutical composition comprises a monoclonal antibody that preferentially binds to an epitope that includes amino acids 36-40 of the Aβ peptide (SEQ ID NO:1). In some embodiments, the antibody binds to amino acids 28-40 of Aβ peptide (SEQ ID NO:1) with an affinity of about 60 nM or less, about 30 nM or less, or about 3 nM or less. In some embodiments, antibody preferentially binds to an epitope that includes amino acid 39 and/or 40 of the Aβ peptide (SEQ ID NO:1) with an affinity of about 60 nM or less, about 30 nM or less, or about 3 nM or less. In some embodiments, antibody preferentially binds to an epitope that includes amino acids 36-40 of the Aβ peptide (SEQ ID NO:1) with an affinity of about 60 nM or less, about 30 nM or less, or about 3 nM or less. In some embodiments, the antibodies of described above do not show significant cross-reactivity with Aβ₁₋₄₁ and/or Aβ₁₋₄₂. In some embodiments, the antibody competitively inhibits binding of a monoclonal antibody comprising the amino acid sequences shown in SEQ ID NO:4 and/or 6, or the monoclonal antibody produced by the hybridoma designated 8A1.2A1. In some embodiments, the antibody binds to the same epitope on Aβ peptide (SEQ ID NO:1) as an antibody comprising amino acid sequence shown in SEQ ID NO:4 or 6, or the monoclonal antibody produced by the hybridoma designated 8A1.2A1 binds. In other embodiments, the pharmaceutical composition comprises a human antibody, a humanized or a chimeric antibody derived from any of the antibodies described herein. Pharmaceutical compositions comprise one or more such compounds and, optionally, a physiologically acceptable carrier. Pharmaceutical compositions within the scope of the present invention may also contain other compounds that may be biologically active or inactive. In a preferred embodiment, the composition comprises at least two antibodies, a first antibody directed against amino acids 16-28 of Aβ peptide and a second antibody directed against amino acids 28-40 of Aβ peptide.

A pharmaceutical composition can contain DNA encoding one or more of the polypeptides as described above, such that the polypeptide is generated in situ. As noted above, the DNA may be present within any of a variety of delivery systems known to those of ordinary skill in the art, including nucleic acid expression systems, bacteria and viral expression systems. Numerous gene delivery techniques are well known in the art, such as those described by Rolland, 1998, Crit. Rev. Therap. Drug Carrier Systems 15:143-198, and references cited therein. Appropriate nucleic acid expression systems contain the necessary DNA sequences for expression in the patient (such as a suitable promoter and terminating signal).

In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., 1989, Proc. Natl. Acad. Sci. USA 86:317-321; Flexner et al., 1989, Ann. N. Y. Acad Sci. 569:86-103; Flexner et al., 1990, Vaccine 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques 6:616-627, 1988; Rosenfeld et al., 1991, Science 252:431-434; Kolls et al., 1994, Proc. Natl. Acad. Sci. USA 91:215-219; Kass-Eisler et al., 1993, Proc. Natl. Acad. Sci. USA 90:11498-11502; Guzman et al., 1993, Circulation 88:2838-2848; and Guzman et al., 1993, Cir. Res. 73:1202-1207. Techniques for incorporating DNA into such expression systems are well known to those of ordinary skill in the art. The DNA may also be “naked,” as described, for example, in Ulmer et al., 1993, Science 259:1745-1749, and reviewed by Cohen, 1993, Science 259:1691-1692. The uptake of naked DNA may be increased by coating the DNA onto biodegradable beads, which are efficiently transported into the cells.

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268 and 5,075,109.

Such compositions may also comprise buffets (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology.

The compositions described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

Therapeutic and Prophylactic Methods

The antibodies (including polypeptides), polynucleotides, and pharmaceutical compositions of the invention can be used in methods for treating, preventing and inhibiting the development of Alzheimer's disease and other diseases associated with altered Aβ or βAPP expression, or accumulation of Aβ peptide, such as Down's syndrome, Parkinson's disease, and multi-infarct dementia. Such methods comprise administering the immunoreactive molecules, antibodies (including polypeptides), polynucleotides or pharmaceutical composition to the subject. In prophylactic applications, pharmaceutical compositions or medicaments are administered to a patient susceptible to, or otherwise at risk of, Alzheimer's disease in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicaments are administered to a patient suspected of, or already suffering from such a disease in amount sufficient to cure, or at least partially arrest, the symptoms of the disease (biochemical, histologic and/or behavioral), including its complications and intermediate pathological phenotypes in development of the disease.

The invention also provides a method of delaying development of a symptom associated with Alzheimer's disease in a subject comprising administering an effective dosage of a pharmaceutical composition comprising an antibody (including polypeptides) or polynucleotide of the invention to the subject. Symptoms associated with Alzheimer disease includes, but not limited to, abnormalities of memory, problem solving, language, calculation, visuospatial perception, judgment, and behavior.

This invention also provides methods of inhibiting or suppressing the formation of amyloid plaques in a subject comprising administering an effective dose of a pharmaceutical composition of the invention to the subject. In some embodiments, the amyloid plaques are in the brain of the subject.

This invention also provides methods of reducing amyloid plaques in a subject comprising administering an effective dose of a pharmaceutical composition of the invention to the subject. In some embodiments, the amyloid plaques are in the brain of the subject.

This invention also provides methods of removing or clearing amyloid plaques in a subject comprising administering an effective dose of a pharmaceutical composition of the invention to the subject. In some embodiments, the amyloid plaques are in the brain of the subject.

This invention also provides methods of reducing Aβ peptide in a tissue (such as brain), inhibiting and/or reducing accumulation of Aβ peptide in a tissue (such as brain), and inhibiting and/or reducing toxic effects of Aβ peptide in a tissue (such as brain) in a subject comprising administering an effective dose of a pharmaceutical composition of the invention to the subject.

The methods described herein (including prophylaxis or therapy) can be accomplished by a single direct injection at a single time point or multiple time points to a single or multiple sites. Administration can also be nearly simultaneous to multiple sites. Frequency of administration may be determined and adjusted over the course of therapy, and is base on accomplishing desired results. In some cases, sustained continuous release formulations of antibodies (including polypeptides), polynucleotides, and pharmaceutical compositions of the invention may be appropriate. Various formulations and devices for achieving sustained release are known in the art.

Patients, subjects, or individuals include mammals, such as human, bovine, equine, canine, feline, porcine, and ovine animals. The subject is preferably a human, and may or may not be afflicted with disease or presently show symptoms. In the case of Alzheimer's disease, virtually anyone is at risk of suffering from Alzheimer's disease if he or she lives long enough. Therefore, the present methods can be administered prophylactically to the general population without the need for any assessment of the risk of the subject patient. The present methods are useful for individuals who do have a known genetic risk of Alzheimer's disease. Such individuals include those having relatives who have experienced this disease, and those whose risk is determined by analysis of genetic or biochemical markers. Genetic markers of risk toward Alzheimer's disease include mutations in the APP gene, particularly mutations at position 717 and positions 670 and 671 referred to as the Hardy and Swedish mutations respectively (see Hardy (1997) Trends Neurosci. 20:154-9). Other markers of risk are mutations in the presenilin genes, PS1 and PS2, and ApoE4, family history of AD, hypercholesterolemia or atherosclerosis. Individuals presently suffering from Alzheimer's disease can be recognized from characteristic dementia, as well as the presence of risk factors described above. In addition, a number of diagnostic tests are available for identifying individuals who have AD. These include measurement of CSF tau and Aβ42 levels. Elevated tau and decreased Aβ42 levels signify the presence of AD. Individuals suffering from Alzheimer's disease can also be diagnosed by ADRDA (Alzheimer's Disease and Related Disorders Association) criteria. In asymptomatic patients, treatment can begin at any age (e.g., 10, 20, 30). Usually, however, it is not necessary to begin treatment until a patient reaches 40, 50, 60 or 70. Treatment typically entails multiple dosages over a period of time. Treatment can be monitored by various ways known in the art over time. In the case of potential Down's syndrome patients, treatment can begin antenatally by administering therapeutic agent to the mother or shortly after birth.

The pharmaceutical composition that can be used in the above methods include, but is not limited to, antibodies that preferentially bind to amino acids 28-40 of Aβ peptide (SEQ ID NO:1), antibodies that preferentially bind to an epitope that includes amino acid 39 and/or 40 of Aβ peptide (SEQ ID NO:1), antibodies that bind to amino acids 28-40 of Aβ peptide (SEQ ID NO:1) with an affinity of about 60 nM or less, about 30 nM or less, or about 3 nM or less, antibodies that preferentially bind to an epitope that includes amino acid 39 and/or 40 of Aβ peptide (SEQ ID NO:1) with an affinity of about 60 nM or less, about 30 nM or less, or about 3 nM or less, and polynucleotides encoding any of the antibodies and polypeptides described herein. In other embodiments, the following antibodies can be used: the antibody that binds to an epitope that includes amino acid 39 and/or 40 of Aβ peptide (SEQ ID NO:1), but does not show significant cross-reactivity with Aβ₁₋₄₂ and Aβ₁₋₄₃ peptide; the Fab fragment of the antibody binds to Aβ₁₋₄₀ peptide (SEQ ID NO:1) with an affinity of about 200 nM or less, or about 1 nM or less; the antibody competitively inhibits binding of a monoclonal antibody comprising ammo acid sequence of SEQ ID NO: 4 and 6 to Aβ₁₋₄₀ peptide (SEQ ID NO:1); the antibody binds to the same epitope to which a monoclonal antibody comprising amino acid sequence of SEQ ID NO: 4 and 6 binds; and antibodies having any combination of the properties described above.

Administration and Dosage

The antibody is preferably administered to the mammal in a carrier; preferably a pharmaceutically-acceptable carrier. Suitable carriers and their formulations are described in Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000. Typically, an appropriate amount of a pharmaceutically-acceptable salt is used in the formulation to render the formulation isotonic. Examples of the carrier include saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of antibody being administered.

The antibody can be administered to the mammal by injection (e.g., systemic, intravenous, intraperitoneal, subcutaneous, intramuscular, intraportal), or by other methods, such as infusion, which ensure its delivery to the bloodstream in an effective form. The antibody may also be administered by isolated perfusion techniques, such as isolated tissue perfusion, to exert local therapeutic effects. Intravenous injection is preferred.

Effective dosages and schedules for administering the antibody may be determined empirically, and making such determinations is within the skill in the art. Those skilled in the art will understand that the dosage of antibody that must be administered will vary depending on, for example, the mammal that will receive the antibody, the route of administration, the particular type of antibody used and other drugs being administered to the mammal. Guidance in selecting appropriate doses for antibody is found in the literature on therapeutic uses of antibodies, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389. A typical daily dosage of the antibody used alone might range from about 1 μg/kg to up to 100 mg/kg of body weight or more per day, depending on the factors mentioned above. Generally, any of the following doses may be used: a dose of at least about 50 mg/kg body weight; at least about 10 mg/kg body weight; at least about 3 mg/kg body weight; at least about 1 mg/kg body weight; at least about 750 ng/kg body weight; at least about 500 μg/kg body weight; at least about 250 ug/kg body weight; at least about 100 μg/kg body weight; at least about 50 μg/kg body weight; at least about 10 ug/kg body weight; at least about 1 μg/kg body weight, or more, is administered.

In some embodiments, more than one antibody may be present. Such compositions may contain at least one, at least two, at least three, at least four, at least five different antibodies (including polypeptides) of the invention.

The antibody may also be administered to the mammal in combination with effective amounts of one or more other therapeutic agents. The antibody may be administered sequentially or concurrently with the one or more other therapeutic agents. The amounts of antibody and therapeutic agent depend, for example, on what type of drugs are used, the pathological condition being treated, and the scheduling and routes of administration but would generally be less than if each were used individually.

Following administration of antibody to the mammal, the mammal's physiological condition can be monitored in various ways well known to the skilled practitioner.

The above principles of administration and dosage can be adapted for polypeptides described herein.

A polynucleotide encoding an antibody (including a polypeptide) of the invention may also be used for delivery and expression of the antibody or the polypeptide in a desired cell. It is apparent that an expression vector can be used to direct expression of the antibody. The expression vector can be administered systemically, intraperitoneally, intravenously, intramuscularly, subcutaneously, intrathecally, intraventricularly, orally, enterally, parenterally, intranasally, dermally, or by inhalation. For example, administration of expression vectors includes local or systemic administration, including injection, oral administration, particle gun or catheterized administration, and topical administration. One skilled in the art is familiar with administration of expression vectors to obtain expression of an exogenous protein in vivo. See, e.g., U.S. Pat. Nos. 6,436,908; 6,413,942; and 6,376,471.

Targeted delivery of therapeutic compositions comprising a polynucleotide encoding an antibody of the invention can also be used. Receptor-mediated DNA delivery techniques are described in, for example, Findeis et al., Trends Biotechnol. (1993) 11:202; Chiou et al, Gene Therapeutics: Methods And Applications Of Direct Gene Transfer J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5, 219,740; 4,777,127; GB Pat. No. 2,200,651; and EP 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0 524 968. Additional approaches are described in Philip, Mol. Cell Biol (1994) 14:2411, and in Woffendin, Proc. Natl. Acad. Sci. (1994) 91:1581.

Diagnostic Uses of the Antibodies

Antibodies of the invention can be used in the detection, diagnosis and monitoring of Alzheimer's disease and other diseases associated with altered Aβ or βAPP expression, such as Down's syndrome. The method comprises contacting a specimen of a patient suspected of having altered Aβ or βAPP expression with an antibody of the invention and determining whether the level of Aβ or βAPP differs from that of a control or comparison specimen.

For diagnostic applications, the antibody typically will be labeled with a detectable moiety including but not limited to radioisotopes, flurescent labels, and various enzyme-substrate labels. Methods of conjugating labels to an antibody are known in the art. In other embodiment of the invention, antibodies of the invention need not be labeled, and the presence thereof can be detected using a labeled antibody which binds to the antibodies of the invention.

The antibodies of the present invention may be employed in any known assay method, such competitive binding assays, direct and indirect sandwich assays, and immunoprecipitation assays. Zola, Monoclonal Antibodies: A Manual of Techniques, pp.147-158 (CRC Press, Inc. 1987).

The antibodies may also be used for in vivo diagnostic assays, such as in vivo imaging. Generally, the antibody is labeled with a radionuclide (such as ¹¹¹In, ⁹⁹Tc, ¹⁴C, ¹³¹I, ¹²⁵I, or ³H) so that the cells or tissue of interest can be localized using immunoscintiography.

The antibody may also be used as staining reagent in pathology, following techniques well known in the art.

Kits

In a further embodiment, the invention provides articles of manufacture and kits containing materials useful for treating pathological conditions such as Alzheimer's disease, Down's syndrome, or other disease associated with altered Aβ or βAPP expression or detecting or purifying Aβ or βAPP. The article of manufacture comprises a container with a label. Suitable containers include, for example, bottles, vials, and test tubes. The containers may be formed from a variety of materials such as glass or plastic. The container holds a composition having an active agent which is effective for treating pathological conditions or for detecting or purifying Aβ or βAPP. The active agent in the composition is an antibody and preferably, comprises monoclonal antibodies specific for Aβ or βAPP. In some embodiments, the active agent is an antibody that binds to an epitope within amino acids 28-40 of Aβ peptide (SEQ ID NO: 1). In some embodiments, the antibody preferentially binds to an epitope that spans amino acids 38-40 of Aβ peptide (SEQ ID NO:1). In some embodiments, the antibody preferentially binds to the amino acids 28-40 of Aβ peptide (SEQ ID NO:1) with an affinity of about 60 nM or less, about 30 nM or less, or about 3 nM or less. In some embodiments, antibody preferentially binds to an epitope that includes amino acid 39 and/or 40 of the Aβ peptide (SEQ ID NO:1) with an affinity of about 60 nM or less, about 30 nM or less, or about 3 nM or less. In some embodiments, the antibody competitively inhibits binding of a monoclonal antibody comprising the amino acid sequences shown in SEQ ID NO:4 and/or 6, or the monoclonal antibody produced by the hybridoma designated 8A1.2A1. In some embodiments, the antibody binds to the same epitope on Aβ peptide (SEQ ID NO:1) as an antibody comprising amino acid sequence shown in SEQ ID NO:4 and/or 6, or the monoclonal antibody produced by the hybridoma designated 8A1.2A1 binds. In some embodiments, the active agent comprises any of the humanized antibody, chimeric antibody or human antibody described herein. The label on the container indicates that the composition is used for treating pathological conditions such as Alzheimer's disease or detecting or purifying Aβ or βAPP, and may also indicate directions for either in vivo or in vitro use, such as those described above.

In some embodiments, the kit of the invention comprises the container described above. In other embodiments, the kit of the invention comprises the container described above and a second container comprising a buffer. It may further include other materials desirable from a commercial and user standpoint, including other buffers, diluents, filters, needles, syringes, and package inserts with instructions for performing any methods described herein (such as methods for treating Alzheimer's disease, and methods for inhibiting or reducing accumulation of Aβ peptide in the brain). In kits to be used for detecting or purifying Aβ or βAPP, the antibody is typically labeled with a detectable marker, such as, for example, a radioisotope, fluorescent compound, bioluminescent compound, a chemiluminescent compound, metal chelator or enzyme.

### EXAMPLES

The following examples are presented to illustrate the present invention and to assist one of ordinary skill in making and using the same. The examples are not intended in any way to otherwise limit the scope of the invention.

### Example 1

### Production and Characterization of Monoclonal Antibodies Directed Against Aβ

The data presented in this example show that high-affinity, specific antibodies directed against Aβ can be generated and provide useful therapeutic agents for targeting Aβ-associated disease. Mice were immunized with 50-100 μg of Aβ₁₋₄₀ peptide in Ribi adjuvant (50 μl per footpad, 100 μl total per mouse) at 10 consecutive weekly intervals as described in Geerligs H J et al., 1989,J. Immunol. Methods 124:95-102; Kenney J S et al., 1989,J. Immunol. Methods 121:157-166; and Wicher K et al., 1989, Int. Arch. Allergy Appl. Immunol. 89:128-135.

Splenocytes were obtained from the immunized mouse and fused with NSO myeloma cells at a ratio of 10:1, with polyethylene glycol 1500. The hybrids were plated out into 96-well plates in DMEM containing 20% horse serum and 2-oxaloacetate/pyruvate/insulin (Sigma), and hypoxanthine/aminopterin/thymidine selection was begun. On day 8, 100 μl of DMEM containing 20% horse serum was added to all the wells. Supernatants of the hybrids were screened by using antibody capture immunoassay. Determination of antibody class was done with class-specific second antibodies.

A panel of monoclonal antibody-producing cell lines was selected for characterization. These cell lines and information describing the corresponding antibodies are listed in Table 2.

TABLE 2
Monoclonal Antibody Characterization

| | Monoclonal | | | | | | |
|---|---|---|---|---|---|---|---|
| Antigen | Producing | | | ELISA | | Affinity | Cross |
| Aβ | Cell Line | Isotype | Epitope | Direct | Capture | Kd (nM) | Reactivity |
| 2286 | 8A1.2A1 | IgG1 | Aβ 28-40 | yes | yes | 2.7 | No |
| 2287 | 11A4.1E5 | IgG2a | Aβ 16-28 | yes | yes | 59 | No |
| 2288 | 23E9.1A1 | IgG2b | Aβ 1-16 | yes | yes | ND | No |
| 2289 | 3C6.1F9 | IgG2b | Aβ 16-28 | yes | yes | 2.9 | No |
| 2290 | 14E10.1F3 | IgG2a | Aβ 16-28 | yes | yes | 9.2 | No |
| 2294 | 13E11.1A12 | IgG2b | Aβ 28-40 | yes | yes | 38 | No |
| 2324 | 10B10.2E6 | IgG2b | Aβ 1-16 | yes | ND | 0.9 | No |

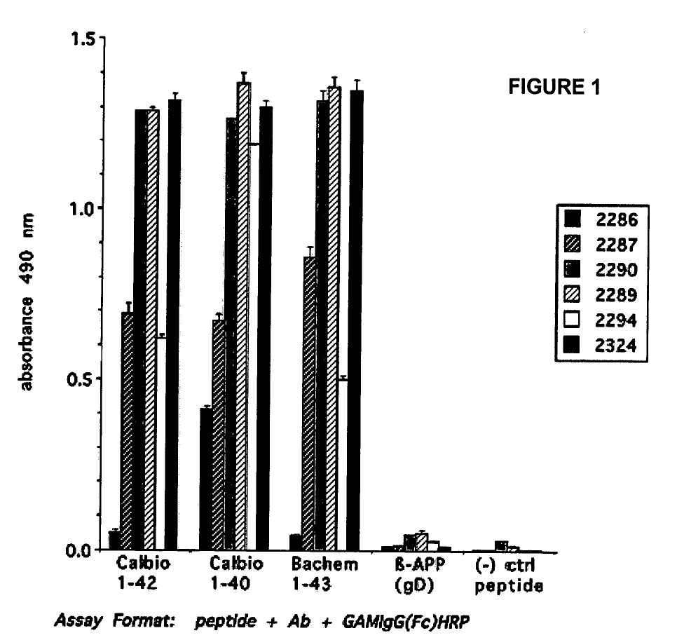

Binding to Aβ from various sources, and to β-APP and a control peptide were tested in a sandwich assay. The Aβ peptides tested were: β-amyloid peptide 1-42 and 1-40, both obtained from Calbiochem (San Diego, Calif.), and β-amyloid peptide 1-43 obtained from Bachem (Torrance, Calif.). Peptide was immobilized onto plates, antibody added, and binding was detected using GAMIgG(Fc)HRP and reading absorbance at 490 nm. Cross-reactivity data, shown in FIG. 1, confirm that nabs directed against Aβ do not cross-react with β-APP.

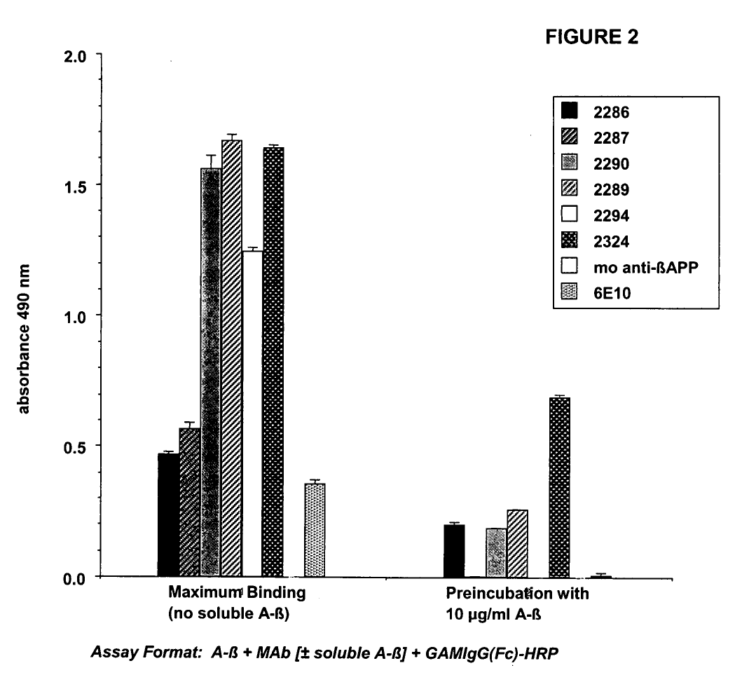

A capture assay was performed to confirm that the antibodies are capable of capturing soluble Aβ peptide. In this assay, Aβ peptide was immobilized onto assay plates, mAB was added either directly or following preincubation with 10 μg/ml Aβ, and binding was detected using GAMIgG(Fc)HRP and reading absorbance at 490 nm. Controls were mouse anti-βAPP and 6E10, a monoclonal antibody that detects amino acid residues 1-17 of human β-amyloid peptide (Signet, Dedham, Mass.). Data demonstrating capture of soluble Aβ are shown in FIG. 2.

Candidate therapeutic antibodies can be assayed ex vivo for their ability to effectively reduce plaque burden in the central nervous system in vivo as described in Bard et al., 200 , Nature Medicine 6(8):916-919.

### Example 2

### Characterization of Epitope on Aβ Polypeptide that Antibodies Directed Against Aβ Bind

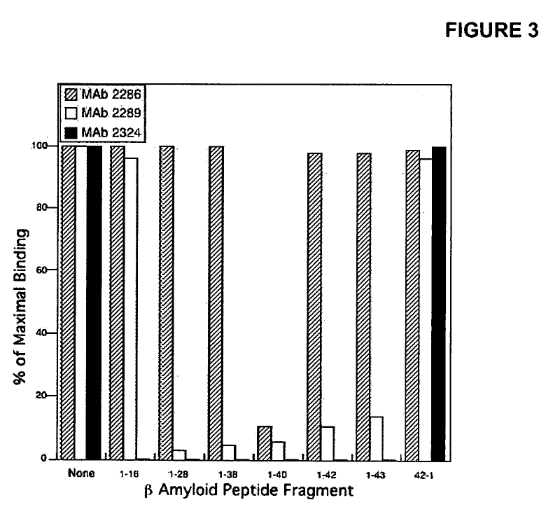

To determine the epitope on Aβ polypeptide that is recognized by the monoclonal antibodies, Surface Plasmon Resonance (SPR, Biacore 3000) binding analysis was used. Aβ₁₋₄₀ polypeptide (SEQ ID NO:1) coupled to biotin (Global Peptide Services, CO) was immobilized on a streptavidin-coated chip. The binding of Aβ antibodies (at 100 nM) to the immobilized Aβ₁₋₄₀ in the absence or presence of different soluble fragments of the Aβ peptide (at 1000 nM, from American Peptide Company Inc., CA). The Aβ peptides which are required to displace binding of monoclonal antibodies 2324, 2289, and 2286 (more precisely antibodies as isolated from their respective hybridoma cell lines 10B10.2E6, 3C6.1F9, and 8A1.2A1) to Aβ₁₋₄₀ were Aβ₁₋₁₆, Aβ₁₋₂₈, and Aβ₁₋₄₀, respectively (FIG. 3). Binding of all three antibodies to Aβ₁₋₄₀ was inhibited by soluble 40. However, the Aβ₁₋₃₈ peptide inhibited the binding of Aβ₁₋₄₀ to MAbs 2324 and 2289, but not to MAb 2286, suggesting that the epitope that MAb 2286 binds includes amino acids 39 and/or 40 of the Aβ₁₋₄₀ peptide (FIG. 3).

In addition, Aβ₁₋₄₂ and Aβ₁₋₄₃ peptide did not inhibit binding of MAb 2286 to Aβ₁₋₄₀ although they could readily inhibit Aβ₁₋₄₀ binding to both MAbs 2324 and 2289 (FIG. 3). These results show that MAb 2286 preferentially binds to Aβ₁₋₄₀, but not to Aβ₁₋₄₂ and Aβ₁₋₄₃.

To further assess the involvement of discrete amino acid residues of the β-amyloid peptide in the binding of Mab 2286, different Aβ₁₋₄₀ variants, in which each of the last 5 amino acids (Aβ₁₋₄₀ amino acid residues 36-40) was individually replaced by an alanine (alanine scanning mutagenesis), were generated by site directed mutagenesis. These Aβ₁₋₄₀ variants (sequences shown in Table 6) were expressed in E. coli as Gluththione-S-Transferase (GST) fusion proteins (Amersham Pharmacia Biotech, Piscataway, N.J. USA) followed by affinity purification on a Glutathione-Agarose beads (Sigma-Aldrich Corp., St. Louis, Mo., USA). As control, Wild-type (WT) Aβ₁₋₄₀ (SEQ ID NO:1) as well as Aβ₁₋₄₁ (SEQ ID NO:13) were also expressed as GST fusion proteins. Aβ₁₋₄₀, Aβ₁₋₄₁ as well as the five different variants (SEQ ID NOS: 14-18) were then immobilized (0.25 μg per well each) onto assay plates and incubated with either Mab 2286 or Mab 2324 (directed to an epitope of Aβ₁₋₄₀ between amino acid 28-40 or 1-16, respectively; each antibody at 2 nM). After 10 consecutive washes, assay plates were incubated with a Biotin-conjugated Goat-anti-Mouse (H+L) antibody (Vector Laboratories, Burllingame Calif., USA) followed by an HRP-conjugated Streptavidin (Amersham Biosciences Corp., N.J., USA). The absorbance of the plate was read at 450 nm.

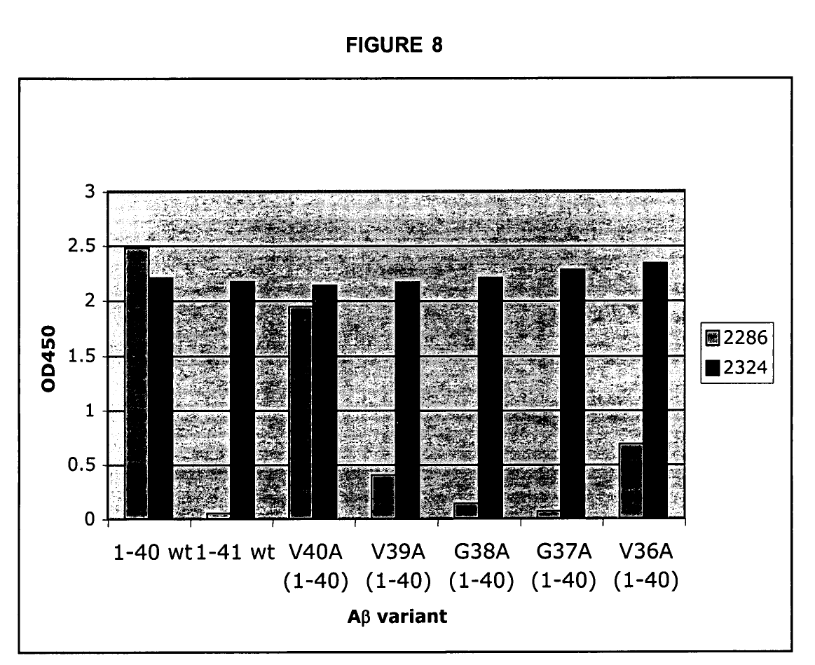

As shown in FIG. 8, Mab 2324 which was directed to a N terminal epitope of Aβ, recognized all variants with the same intensity and served as internal positive control of protein concentration and protein integrity on the plate. Mab 2286 did not recognize Aβ₁₋₄₁ (or Aβ₁₋₄₂ as shown in FIG. 3) while mutation to Ala of the C-terminal V40 did not affect binding, suggesting that the amino carboxy terminal moiety of the protein might be directly involved in Mab 2286 epitope while the side chain of V40 might be less important. Aβ₁₋₄₀ variants V39A, G38A, G37A and V36A showed reduced binding to Mab 2286, demonstrating that Mab 2286 epitope extended for at least 5 amino acids at the C terminal end of Aβ₁₋₄₀. Mutations of V and G to A ate very conservative and are not likely to produce important conformational changes in proteins, therefore, the large effect of these mutations to Mab 2286 binding might be due to the ability of the antibody to differentiate between the mentioned amino acids in the context of Aβ and these data demonstrated a very high degree of specificity for this antibody.

### Example 3

### Production and Characterization of Monoclonal Antibodies Directed Against βAPP

Mice were immunized with APP as described in Example 1. A panel of monoclonal antibody-producing cell lines was selected for characterization. These cell lines and information describing the corresponding antibodies are listed in Table 3.

TABLE 3
Monoclonal Antibody Characterization

| | Monoclonal | | | | Affinity | Cross |
|---|---|---|---|---|---|---|
| Antigen | Producing | | ELISA | | Kd | Reac- |
| APP | Cell Line | Isotype | Direct | Capture | (nM) | tivity |
| 2312 | 25E12.1F9.1H8 | IgG1 | yes | ND | ND | No |
| | (BP26) | | | | | |
| 2313 | 24H4.2E10.1F5 | IgG1 | yes | ND | ND | No |
| | (BP27) | | | | | |
| 2334 | 1F10.8E6.2A2 | IgG2b | yes | ND | ND | No |
| | (BP80) | | | | | |
| 2335 | 13E12.1C5 | IgG2b | yes | ND | ND | No |
| | (BP81) | | | | | |
| 2336 | 14D9.1G8 | IgG1 | yes | ND | ND | No |
| | (BP82) | | | | | |

### Example 4

### Humanization of Monoclonal Antibody 2286

The mouse antibody 2286 was humanized by grafting heavy chain CDRs (Kabat and/or Chothia) into the human germline acceptor sequence VH3 and VH4; and the light chain Kabat CDRs were grafted into the human germline acceptor sequence 08. The humanized heavy chain and light chain of antibody 2286 are shown in Table 4 below.

TABLE 4
Amino acid sequences of the heavy and light chains of the humanized antibodies

Humanized 2286 Light Chain Variable Domain (germline framework O8)

| | |
|---|---|
| DIQMTQSPSSLSASVGDRVTITCSASQGISNYLNWYQQKPGKAPKLLIYYTSSLHSG | (SEQ ID NO: 13) |
| VPSFPSRFSGSGSGTDFTFTISSLQPEDJATYYCQQYRKLPYTFGGGTKVEIIKR | |

Humanized 2286 Heavy Chain Variable Domain (germline framework VH3)

| | |
|---|---|
| EVQLVESGGGLVQPGGSLRLSCAASgfdfsrYWMNWVRQAPGKGLEWVSEINPDSS | (SEQ ID NO: 14) |
| TINYTPSLKDRFTISRDNAKNTLYLQMNSLRAEDTAVYYCARQMGYWGQGTTLT | |
| VSS | |

Humanized 2286 Heavy Chain Variable Domain (germline framework VH4)

| | |
|---|---|
| QVQLQESGPGLVKPSETLSLTCTVSgfdfsrYWMNWIRQPPGKGLEWIGEINPDSSTIN | (SEQ ID NO: 15) |
| YTPSLKDRVTISKDTSKNQFSLKLSSVTAADTAVYYCARQMGYWGQGTLVTvSS | |

*CDR boundaries in antibodies heavy and light chains were determined according to the Kabat nomenclature (marked by an underline) except for CDRH1 where both Kabat and Chothia (lower case) nomenclature were used to define CDR boundaries.

Equilibrium dissociation constant (K_{D}) values of anti-Aβ Fab fragments of monoclonal antibodies were determined by the ‘Steady-State’ method using the BIAcore3000™ surface plasmon resonance (SPR) system (BIAcore, INC, Piscaway N.J.) described above. Kinetic association rates (kₒₙ) and dissociation rates (k_{off}) were obtained simultaneously by fitting the data to a 1:1 Langmuir binding model (Lofas & Johnsson, 1990) using the BIAevaluation program calculated. The affinity of humanized antibodies and mouse antibody 2286 are shown in Table 5 below.

TABLE 5
Binding affinity of humanized antibodies

| | k_{off} (S⁻¹) | kₒₙ (M⁻¹, S⁻¹) | K_{D} (nM) |
|---|---|---|---|
| Mouse 2286 Fab | 0.044 | 5.8 × 10⁴ | 76 |
| Humanized 2286 | nd | nd | 500 |
| Fab (O8; VH3) | | | |
| Humanized 2286 | nd | nd | 500 |
| Fab (O8; VH4) | | | |

### Example 5

### Antibodies Directed Against Aβ Peptide Reduce Histological Symptoms in an Animal Model of Alzheimer's Disease

This example demonstrates that the monoclonal antibodies of the invention provide an effective therapeutic agent for the treatment and prevention of Alzheimer's disease. Surprisingly, these data show that antibodies directed at the C terminus (i.e. aa 28-40) of Aβ were just as effective at clearing Aβ, thioflavine-S, and increasing MHC-II staining as antibodies directed at the N terminus (aa 1-16) in this mouse model of Alzheimer's disease. Because antibodies targeting the N terminus of Aβ are likely advantageous due to increased ability to recognize the precursor and/or disrupt aggregation of amyloid deposits, these results provide a promising new therapeutic strategy for the treatment of Alzheimer's disease.

To evaluate the therapeutic effects of anti Aβ antibodies in vivo, monoclonal antibodies 2324, 2286, and 2289, more precisely, antibodies as isolated from their respective hybridoma cell lines 10B10.2E6, 8A1.2A1, and 3C6.1F9, were injected to transgenic mice over-expressing the ‘Swedish’ mutant amyloid precursor protein (APP; Tg2576; K670N/M671L; Hsiao et al., 1996, Science 274:99-102). The Alzheimer's-like phenotype present in these mice has been well-characterized (Holcomb L A et al., 1998, Nat. Med. 4:97-100; Holcomb L A et al., 1999, Behav. Gen. 29:177-185; and McGowan E, 1999, Neurobiol. Dis. 6:231-244).

In terms of the experimental procedure followed, which is not necessary for describing or enabling the invention, antibodies were injected intracranially to Tg2576 transgenic mice of 16 months of age. Injected antibodies were monoclonal antibodies 2324 (at 1.2 μg in a volume of 2 μl) , 2286 (at 2 μg in a volume of 2 μl) and 2289 (at 2 μg in a volume of 2 μl) and a control monoclonal antibody directed against a Drosophila protein termed “Amnesiac” (at 2 μg in a volume of 2 μl), more precisely, antibodies as isolated from their respective hybridoma cell lines 10B10.2E6, 8A1.2A1, and 3C6.1F9, were injected intracranially. Histopathology of the mice frontal cortex and hippocampus were evaluated at 3 days after injection. Three-day time point was chosen from time course work with another antibody indicating that the amyloid clearance was complete by that interval and the microglial activation was maximal compared to 1 day or 7 days. Data were presented as the ratio of injected side to non-injected side for Aβ, thioflavine-S and MHC-II staining.

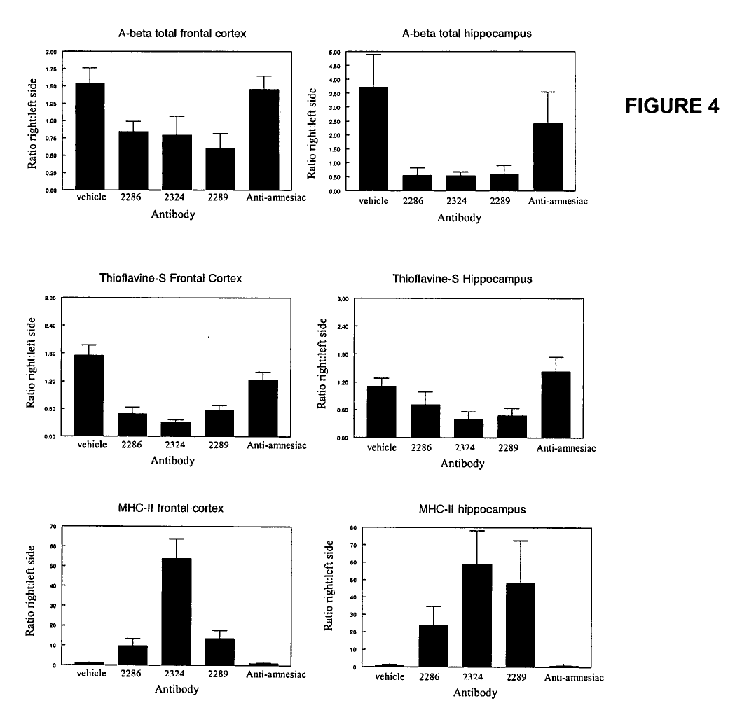

Although the three antibodies used in the study are directed to different parts of the Aβ peptide (amino acids 1-16, 16-28, and 28-40 respectively), all removed both Aβ deposits and thioflavine-S deposits (the latter detects the toxic fibrillar form of Aβ deposits) in hippocampus and cortex by a considerable percentage (FIG. 4, by 40-80%) compared with control groups (anti-amnesiac antibody and vehicle injected groups). They also activated microglia, as evaluated by MHC-II staining (FIG. 4). There was no consistent difference between the three Aβ antibodies in their capacity to remove Aβ, thioflavine-S, or increase MHC-II staining in these mice. These results were unexpected given the previously published studies that indicated that N terminally (i.e. aa 1-16)- but not C terminally (i.e. aa 28-40)-directed antibodies were important for AB deposit clearance. (Solomon, B. et al., 1996, Proc. Natl. Acad. Sci. USA 93:452-455; Solomon, B. et al., 1997, Proc. Natl. Acad. Sci. USA 94:4109-4112.)

### Example 6

### Potential Role of the Fc Domain of Antibody 2286 in Microglial Activation and Amyloid Clearance

To investigate the potential role of the Fc domain of anti-Aβ antibody 2286 in microglial activation and amyloid clearance, the effect of F_{(ab′)2} fragments of antibody 2286, the intact antibody, and a control monoclonal antibody directed against the drosophila protein amnesiac in microglial activation and amyloid clearance were compared in an animal model described in Example 6, where antibody is administered intracranially.

Preparation of F_{(ab′)2} Fragments:

F_{(ab′)2} fragments from anti-Aβ monoclonal antibody 2286, and a control monoclonal antibody directed against the drosophila protein amnesiac were prepared using the Immunopure IgG1 F_{ab} and F_{(ab′)2} preparation kit (Pierce Biotechnology, Rockford, Ill.). The instructions provided with the kit were followed. Briefly, 0.5 ml of 1 mg/ml IgG was added to 0.5 ml mouse IgG1 mild elution buffer. This was applied to an equilibrated immobilized ficin column, allowed to enter the column and digested at 37° C. for 20 hours. A 4 ml elution was obtained and applied to an equilibrated immobilized protein A column for separation of the F_{(ab′)2} from Fc fragments and undigested IgG. Four 1 ml fractions of product were obtained. As determined by running a gel electrophoresis only the 2^{nd} and 3^{rd} elutions were found to contain F_{(ab′)2} fragments and appeared of similar intensities on the gel. The two elutions containing F_{(ab′)2} fragments were pooled and concentrated using Centricon centrifugal filter devices (Millipore Corp. Bedford, Mass.) to a volume of approximately 200 μl. Preliminary experiments found that injections of the F_{(ab′)2} fractions concentrated directly from the column caused seizures when injected into some mice. Thus the initial concentrate was diluted in 4 ml of fresh PBS and reconcentrated to dilute residual proprietary elution buffer components which may cause seizures. No seizures or neurotoxicity were found in the mice included here. The concentrated product was run on an SDS-polyacrylamide-gel electrophoresis (SDS-PAGE). A Bradford assay was also performed to establish concentrations of the F_{(ab′)2} fragments using Bradford protein assay reagent concentrate (Bio-Rad, Hercules, Calif.).

F_{(ab′)2} fragments prepared from anti-Aβ monoclonal antibody 2286, and a control monoclonal antibody directed against the drosophila protein amnesiac were analyzed via SDS-polyacrylamide-gel electrophoresis (PAGE). The gel showed very pure product with no contamination by undigested IgG or Fc fragments, with a single band at approximately 105 kDa, the molecular weight for F_{(ab′)2} fragments. The intact IgG molecule produced one intense band at approximately 150 kDa, the correct molecular weight for IgG molecules and a less intense band at approximately 110 kDa. Following confirmation of purity via SDS-PAGE we then performed a Bradford assay to assess the recovery of F_{(ab′)2} in the purified fraction. Because we dissolved the anti-Aβ F_{(ab′)2} fragments in a smaller volume than was used for the starting material the concentration of F_{(ab′)2} fragments injected intracranially was 1.2 μg/μl, while the holoantibody concentration was 1 μg/μl, resulting in an excess of anti-Aβ Fv domains in the F_{(ab′)2} solutions.

Antibody Fractions Study

Twenty Tg2576 APP transgenic mice aged 19.5 months were assigned to one of four groups, all groups received intracranial injections into the frontal cortex and hippocampus. The first group received anti-Aβ antibody 2286 at a concentration of 2 μg /2 μl in each region. The second group received anti-Aβ F_{(ab′)2} fragments prepared from the anti-Aβ antibody 2286 at 2.2 μg/2 μl in each region. The third group received IgG directed against drosophila amnesiac protein as a control for nonspecific aspects of intact IgG injection. The final group received control F_{(ab′)2} fragments prepared from the IgG directed against drosophila amnesiac protein to control for nonspecific effects of F_{(ab′)2} injection. All mice survived for 72 hours after surgery.

Surgical Procedure:

On the day of surgery the mice were weighed, anesthetized with isoflurane and placed in a stereotaxic apparatus (51603 dual manipulator lab standard, Stoelting, Wood Dale, Ill.). A midsagittal incision was made to expose the cranium and two burr holes were drilled using a dental drill over the right frontal cortex and hippocampus to the following coordinates: Cortex: AP+1.5 mm, L−2.0 mm, hippocampus: A−2.7 mm, L−2.5 mm, all taken from bregma. A 26 gauge needle attached to a 10 μl Hamilton (Reno, Nev.) syringe was lowered 3 mm ventral to bregma and a 2 μl injection was made over a 2 minute period. The incision was cleaned with saline and closed with surgical staples.

Tissue Preparation:

On the day of sacrifice mice were weighed, overdosed with 100 mg/kg pentobarbital (Nembutal sodium solution, Abbott laboratories, North Chicago Ill.) and intracardially perfused with 25 ml 0.9% sodium chloride followed by 50 ml freshly prepared 4% paraformaldehyde (pH-7.4). Brains were rapidly removed and immersion fixed for 24 hours in freshly prepared 4% paraformaldehyde. The brains were then incubated for 24 hours in 10, 20 and 30% sucrose sequentially to cyroprotect them. Horizontal sections of 25 μm thickness were then collected using a sliding microtome and stored at 4° C. in DPBS buffer with sodium azide to prevent microbial growth.

Immunohistochemical Methods:

Six to eight sections approximately 100 μm apart were selected spanning the injection site and stained using free-floating immunohistochemistry methods for total Aβ (rabbit antiserum primarily reacting with the N-terminal of the Aβ peptide 1:10000) and CD45 (Serotec, Raleigh N.C., 1:3000) as previously described (Gordon et al., 2002). For immunostaining, some sections were omitted from the primary antibody to assess non-specific immunohistochemical reactions. Adjacent sections were mounted on slides and stained using 4% thioflavine-S (Sigma-Aldrich, St Louis Mo.) for 10 minutes. It should be noted that there were a limited number of sections that include the injection volume. The procedure followed was to measure a few markers reliably rather than a larger number of markers with fewer sections each.

Data Analysis:

The immunohistochemical reaction product on all stained sections was measured using a videometric V150 image analysis system (Oncor, San Diego, Calif.) in the injected area of cortex and hippocampus and corresponding regions on the contralateral side of the brain. Data were presented as the ratio of injected side to non-injected side for Aβ, thioflavine-S and CD45. Normalizing each injection site to the corresponding contralateral site diminishes the influence of interanimal variability and permits reliable measurements of drug effects with a smaller number of mice. To assess possible treatment-related differences, the ratio values for each treatment group were analyzed by ANOVA using StatView software version 5.0.1 (SAS Institute Inc., NC) followed by Fischer's LSD means comparisons.

Results:

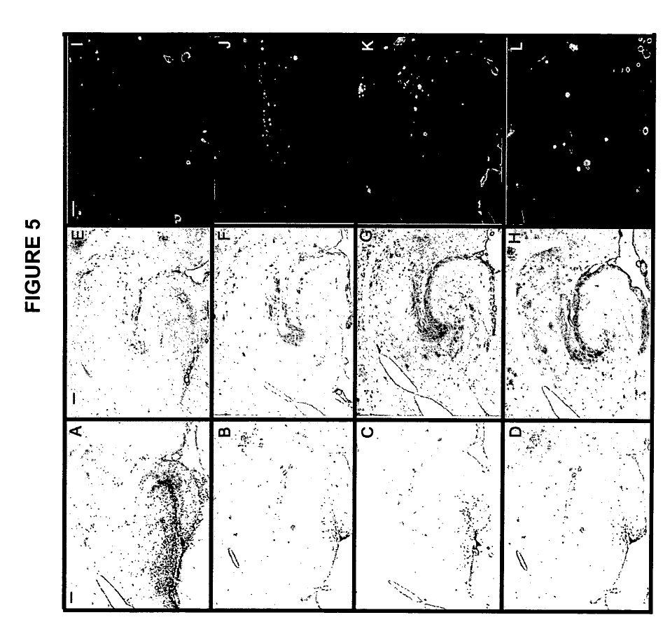

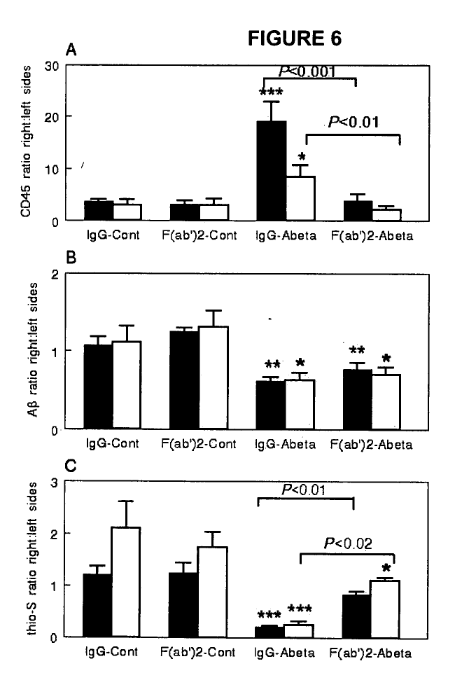

The only antibody which activated microglia 72 hours following intracranial injection into frontal cortex and hippocampus was the intact anti-Aβ antibody 2286. The frontal cortex showed a greater degree of activation than the hippocampus, however, in both regions the activation was significantly greater than that in the groups receiving control anti-amnesiac protein IgG, F_{(ab′)2}, or anti-Aβ 2286 F_{(ab′)2} (FIG. 5A, C and D, FIG. 6A; P<0.01 or greater in all comparisons). The pattern of activation in the hippocampus following the anti-Aβ antibody 2286 injection resembled the pattern when using the anti-Aβ antibody 44-352, a monoclonal antibody that binds to beta-amyloid amino acids 1-16 (Biosource, Camarillo, Calif.). There was a very intense area of activation in the granule cell layer of the dentate gyrus, with a much more diffuse activation filling the remainder of the dentate gyrus (FIG. 5A). Interestingly, the anti-Aβ F_{(ab′)2} fragments produced no microglial activation in both the frontal cortex and hippocampus (FIG. 5B, FIG. 6A).

Aβ immunohistochemistry in the two anti-amnesiac protein control groups showed the typical staining pattern observed in APP transgenic mice 19.5 months (FIG. 5G and H). This pattern was qualitatively the same as observed at 16 months, although quantitatively greater as the mice were 3.5 months older. Both the anti-Aβ antibody and the anti-Aβ F_{(ab′)2} groups significantly reduced total Aβ immunohistochemistry to a similar extent 72 hours following injection into frontal cortex and hippocampus. In the frontal cortex there was a reduction of approximately 60% (FIG. 6B). In the hippocampus the reduction was approximately 65% (FIG. 5E and F, FIG. 6B).

Thioflavine-S staining detects only compact fibrillar amyloid deposits. The mice receiving intracranial injections of either control anti-amnesiac protein IgG or control F_{(ab′)2} resembled the typical staining observed in the APP transgenic mouse at this age. In the hippocampus the majority of thioflavine-S positive plaques were located in the outer molecular layer of Ammon's horn and the dentate gyrus near the hippocampal fissure (FIG. 5K and L). Anti-Aβ antibody IgG significantly reduced thioflavine-S positive compact plaque by approximately 90% in the frontal cortex and hippocampus (FIG. 6C). There were no, or very few, remaining thioflavine-S positive deposits in the hippocampus (FIG. 5I). In contrast, the anti-Aβ F_{(ab′)2} fragments did not remove compact amyloid plaques as effectively as the whole IgG molecule. In the frontal cortex there was no significant reduction in thioflavine-S staining when compared to either control antibody group (FIG. 6C). In the hippocampus there was a significant difference between the anti-Aβ F_{(ab′)2} group and the control groups (P<0.05), however, this reduction was also significantly less than the reduction observed with the whole IgG molecule (FIG. 5J, FIG. 6C; P<0.02 or greater).

As expected, when CD45 and thioflavine-S values for all groups receiving anti-Aβ IgG or anti-Aβ F_{(ab′)2}, regardless of subsequent treatments, were compared in a single large regression analysis, there was a significant correlation between increasing levels of microglial activation as detected by CD45 immunohistochemistry and compact plaque removal as detected by thioflavine-S staining in the frontal cortex when log transformed CD45 values were used (P<0.001, R=0.57). This correlation was also observed in the hippocampus (P<0.02, R=0-427).

### Example 7

### Increased Serum Aβ Concentration Following Peripheral Injection of Antibody 2286

This experiment was performed to test the efficacy of monoclonal antibody 2286 following systemic passive immunization of a transgenic mouse model for Alzheimer's disease. Tg2576 transgenic mice (Hsiao et al., 1996, Science 274:99-102) that were 19 months of age were injected intraperitoneally (IP) with either monoclonal antibody 2286 or an anti-amnesiac antibody (IgG1 control). Antibodies were injected once every week at a dose of 10 mg per Kg of body weight for periods of one, two or three months after which both Aβ serum concentrations as well as titers of anti-Aβ antibodies in the serum were measured.

Serum concentrations of Aβ were determined by using a capture assay, in which an anti-Aβ antibody (Clone 6E10, Signet Laboratories Inc., Dedham, Mass.) was immobilized onto assay plates and incubated with diluted serum samples derived from the treated mice. After 10 consecutive washes, assay plates were incubated with a second Biotin-conjugated anti-Aβ antibody (Clone 4G8, Signet Laboratories Inc., Dedham Mass., USA) followed by addition of an HRP-conjugated Streptavidin (Amersham Biosciences Corp., N.J., USA). The absorbance at 450 nm of the assay plates was determined and concentrations of Aβ in the serum samples were determined by normalizing with known concentrations of synthetic Aβ1-40 (American Peptide Company Inc., Sunnyvale Calif., USA) as standards. To measure 2286 antibody titers in the serum samples, antibody- Aβ complexes were dissociated by low pH and were incubated with assay plates that were pre-coated with synthetic Aβ₁₋₄₀ (0.25 μg per well each). After 10 consecutive washes, assay plates were incubated with a Biotin-conjugated Goat-anti-Mouse (H+L) antibody (Vector Laboratories, Burllingame Calif., USA) followed by an HRP-conjugated Streptavidin (Amersham Biosciences Corp., N.J., USA), and absorbances at 450 nm were measured. Concentrations of anti-Aβ antibodies were calculated from a standard curve that was generated by performing the same assay with known concentration of affinity purified 2286.

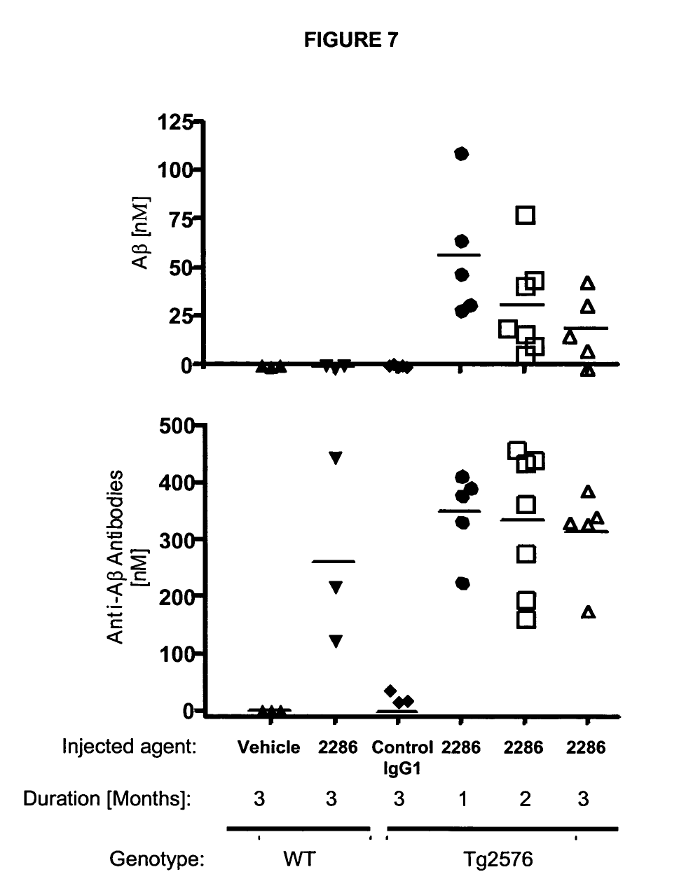

As shown in FIG. 7, rapid increase in serum Aβ concentration was observed following peripheral administration of 2286 but not anti-amnesiac antibodies to Tg2576 mice. Titers of anti-Aβ antibody in serum samples showed significant positive correlation between antibody concentration in serum and serum Aβ concentration in treated transgenic mice (r²=0.5125, F=26.28, P<0.0001, data analyzed by INSTAT PRISM v.4, GraphPad Software Inc., San Diego, Calif.). These data suggest that monoclonal antibody 2286 may have changed Aβ equilibrium between CNS and plasma, and administration of monoclonal antibody 2286 may facilitate clearance of Aβ out of the CNS. To test this possibility, brain amyloid burden would be measured to determine whether an increase of Aβ concentration in serum correlates with a decrease of brain amyloid burden in treated mice.

TABLE 6
Amino acid sequences of beta amyloid peptides and variants

| | | |
|---|---|---|
| 1-40 | DAEFRHDSGYEVHHQKLVFFAEDVGSNKGAIIGLMVG | (SEQ ID NO:1) |
| (WT) | GVV | |
| 1-42 | DAEFRHDSGYEVHHQKLVFFAEDVGSNKGAIIGLMVG | (SEQ ID NO:11) |
| (WT) | GVVIA | |
| 1-43 | DAEFRHDSGYEVHHQKLVFFAEDVGSNKGAIIGLMVG | (SEQ ID NO:12) |
| (WT) | GVVIAT | |
| 1-41 | DAEFRHDSGYEVHHQKLVFFAEDVGSNKGAIIGLMVG | (SEQ ID NO:13) |
| (WT) | GVVI | |
| V36A | DAEFRHDSGYEVHHQKLVFFAEDVGSNKGAIIGLMAG | (SEQ ID NO:14) |
| (1-40) | GVV | |
| G37A | DAEFRHDSGYEVHIHQKLVFFAEDVGSNKGMIGLMVA | (SEQ ID NO:15) |
| (1-40) | GVV | |
| G38A | DAEFRHDSGYEVHHQKLVFFAEDVGSNKGAIIGLMVG | (SEQ ID NO:16) |
| (1-40) | AVV | |
| V39A | DAEFRHDSGYEVHHQKLVFFAEDVGSNKGAIIGLMVG | (SEQ ID NO:17) |
| (1-40) | GAV | |
| V40A | DAEFRHDSGYEVHHQKLVFFAEDVGSNKGAIIGLMVG | (SEQ ID NO:18) |
| (1-40) | GVA | |

TABLE 7

Homo sapiens amyloid beta (A4) precursor protein (APP) (SEQ ID NO:2):
MLPGLALLLLAAWTARALEVPTDGNAGLLAEPQIAMFCGRLNMHMNVQNGKWD
SDPSGTKTCIDTKEGILQYCQEVYPELQITNVVEANQPVTIQNWCKRGRKQCKTHP
HFVIPYRCLVGEFVSDALLVPDKCKELHQERMDVCETHLHWHTVAKETCSEKSTN
LHDYGMLLPCGJDKIFRGVEFVCCPLAEESDNVDSADAEEDDSDVWWGGADTDY
ADGSEDKVVEVAEEEEVAEVEEEEADDDEDDEDGDEVEEEAEEPYEEATERTTSA
TTTTTTTESVEEVVREVCSEQAETGPCRAMISRWYFDVTEGKCAPFFYGGCGGNR
NNFDTEEYCMAVCGSAMSQSLLKTTQEPLARDPVKLPTTAASTPDAVDKYLETPG
DENEHAHFQKAKERLEAKHRERMSQVMREWEEAERQAKNLPKADKKAVIQHFQ
EKVESLEQEAANERQQLVETHMARVEAMLNDRRRLALENYITALQAVPPRPRHVF
NMLKIKYVRAEQKDRQHTLKHFEHVRMVDPKKAAQRSQVMTHLRVJYERMNQS
LSLLYNVPAVAEEIQDEVDELLQKEQNYSDDVLANMISEPRISYGNDAIMPSLTET
KTTVELLPVNGEFSLDDLQPWHSFGADSVPANTENEVEPVDARPAADRGLTTRPG
SGLTNIKTEEISEVKMDAEFRHDSGYEVHHQKLVFFAEDVGSNKGAIIGLMVGGV
VIATVIVITLVMLKXKQYTSThIIHGVVEVDAAVTPEERHLSKMQQNGYENPTYKFF
EQMQN

TABLE 8
Monoclonal Antibody 2286 Nucleic Acid Sequence:

Heavy Chain [variable domain and constant domain 1 (CH1)]; SEQ ID NO: 3:
gaggtgaagcttctcgagtctggaggtggcctggtgcagcctggaggatccctgaaactctcctgtgcagcctcaggattcgatttt
agtagatactggatgaattgggtccggcaggctccagggaaagggctagaatggattggagaaattaatccagatagcagtacga
taaactatacgccatctctaaaggataaattcatcatctccagagacaacgccaaaaatacgctgtacctgcaaatgagcaaagtga
gatctgaggacacagccctttattactgtgcaagacaaatgggctactggggccaaggcaccactctcacagtctcctcagccaaa
acgacacccccatctgtctatccactggcccctggatctgctgcccaaactaactccatggtgaccctgggatgcctggtcaaggg
ctatttccctgagccagtgacagtgacctggaactctggatccctgtccagcggtgtgcacaccttcccagctgtcctgcagtctgac
ctctacactctgagcagctcagtgactgtcccctccagcacctggcccagcgagaccgtcacctgcaacgttgcccacccggcca
gcagcaccaaggtggacaagaaaattgtgcccagggattgt
Light Chain; SEQ ID NO: 5:
gatatccagatgacacagactacatcctccctgtctgcctctctgggagacagagtcaccatcagttgcagtgcaagtcagggcatt
agcaattatttaaactggtttcagcagaaaccagatggaactgttaaactcctgatctattacacatcaagtttacactcaggagtccca
tcaaggttcagtggcagtgggtctgggacagattattctctcaccatcagcaacctggaacctgaagatattgccacttactattgtca
gcagtataggaagcttccgtacacgttcggaggggggaccaagctggaaataaaacgggctgatgctgcaccaactgtatccatc
ttcccaccatccagtgagcagttaacatctggaggtgcctcagtcgtgtgcttcttgaacaacttctaccccaaagacatcaatgtcaa
gtggaagattgatggcagtgaacgacaaaatggcgtcctgaacagttggactgatcaggacagcaaagacagcacctacagcat
gagcagcaccctcacgttgaccaaggacgagtatgaacgacataacagctatacctgtgaggccactcacaagacatc~cttca
cccattgtcaagagcttcaacaggaatgagtgt

TABLE 9
Monoclonal Antibody 2286 Amino Acid Seciuence:

Heavy Chain [variable domain and constant domain 1 (CH1)]; SEQ ID NO: 4:
EVKLLESGGGLVQPGGSLKISCAASGFDFSRYWMNWVRQAPGKGLEWIGEINPDS
STINYTPSLKDKFIISRDNAKNTLYLQMSKVRSEDTAIYYCARQMGYWGQGTTLT
VSSAKTTPPSVYPLAPGSAAQTNSMVTLGCLVKGYFPEPVTVTWNSGSLSSGVHTF
PAVLQSDLYTLSSSVTVPSSTWPSETVTCNVAHPASSTKVDKKIVPRDC
Light Chain; SEQ ID NO: 6:
DIQMTQTTSSLSASLGDRVTISCSASQGISNYLNWFQQKPDGTVKLLIYYTSSLHSG
VPSRFSGSGSGTDYSLTISNLEPEDIATYYCQQYRKLPYTFGGGTKLEIKRADAAPT
VSIFPPSSEQLTSGGASVVCFLNNEYPKDINVKWKIDGSERQNGVLNSWTDQDSKD
STYSMSSTLTLTKDEYERHNSYTCEATHKTSTSPIVKSFNRNEC

TABLE 10
Monoclonal Antibody 2324 Nucleic Acid Sequence:

Heavy Chain [variable domain and constant domain 1 (CH1)]; SEQ ID NO: 7:
gttactctgaaagagtctggccctgggatattgaagccctcacagaccctcagtctgacttgttctttctctgggttttcactgagcactt
ctggtatgggtgtaggctggattcgtcagtcttcagggaagggtctggagtggctggcacacatttggtgggatgatgataagtact
ataacccatccctgaagagccagctcacaatctccaaggatacctccagaaaccaggtattcctcaagatcaccagtgtggacact
gcagatactgccacttactactgtgctcgaaggggggtacgacatagagactactttgactactggggccaaggcaccactctcac
agtctcctcagccaaaacaacacccccatcagtctatccactggcccctgggtgtggagatacaactggttcctccgtgactctggg
atgcctggtcaagggctacttccctgagtcagtgactgtgacttggaactctggatccctgtccagcagtgtgcacaccttcccagct
ctcctgcagtctggactctacactatgagcagctcagtgactgtcccctccagcacctggccaagtcagaccgtcacctgcagcgtt
gctcacccagccagcagcaccacggtggacaaaaaacttgagcccagcgggcccatttcaacaatcaacccc
Light Chain; SEQ ID NO: 9:
gatgttttgatgacccaaactccactctccctgcctgtcagtcttggagatcaagcctccatctcttgcagatctagtcagagcattgta
catagtaatggaaacacctatttagaatggtacctgcagaaaccaggccagtctccaaaactccttatctacaaagtttccaaccgatt
ttctggggtcccagacaggttcagtggcagtggatcagggacagatttcacactcaagatcagcagagtggaggctgaggatctg
ggagtttattactgctttcaaggttcacgtgttcctctcacgttcggtgctgggaccaagctggagctgaaacgggctgatgctgcac
caactgtatccatcttcccaccatccagtgagcagttaacatctggaggtgcctcagtcgtgtgcttcttgaacaacttctaccccaaa
gacatcaatgtcaagtggaagattgatggcagtgaacgacaaaatggcgtcctgaacagttggactgatcaggacagcaaagaca
gcacctacagcatgagcagcaccctcacgttgaccaaggacgagtatgaacgacataacagctatacctgtgaggccactcacaa
gacatcaacttcacccattgtcaagagcttcaacaggaatgagtgt

TABLE 11
Monoclonal Antibody 2324 Amino Acid Sequence:

Heavy Chain [variable domain and constant domain 1 (CH1)]; SEQ ID NO: 8:
VTLKESGPGILKPSQTLSLTCSFSGFSLSTSGMGVGWRQSSGKGLEWLAHIWWDD
DKYYNPSLKSQLTISKDTSRNQVFLKITSVDTADTATYYCARRGVRHRDYFDYWG
QGTTLTVSSAKTTPPSVYPLAPGCGDTTGSSVTLGCLVKGYFPESVTVTWNSGSLS
SSVHTFPALLQSGLYTMSSSVTVPSSTWPSQTVTCSVAHPASSTTVDKKLEPSGPIS
TINP
Light Chain; SEQ ID NO: 10:
DVLMTQTPLSLPVSLGDQASISCRSSQSIVHSNGNTYLEWYLQKPGQSPKLLJYKVS
NRFSGVPDRFSGSGSGTDFTLKISRVEAEDLGVYYCFQGSRVPLTFGAGTKILELKR
ADAAPTVSIFPPSSEQLTSGGASVVCFLNNFYPKDTNVKWKIDGSERQNGVLNSWT
DQDSKDSTYSMSSTLTLTKDEYERHNSYTCEATHKTSTSPIVKSFNRNEC

Deposit of Biological Material

The following materials have been deposited with the American Type Culture Collection, 10801 University Boulevard, Manassas, Va. 20110-2209, USA (ATCC):

| Material | Antibody No. | ATCC Accession No. | Date of Deposit |
|---|---|---|---|
| 8A1.2A1 | 2286 | PTA-5199 | May 15, 2003 |

This deposit was made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit The deposit will be made available by ATCC under the terms of the Budapest Treaty, and subject to an agreement between Rinat Neuroscience Corp. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC Section 122 and the Commissioner's rules pursuant thereto (including 37 CFR Section 1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

What is claimed is:

1. A method for treating Alzheimer's disease comprising administering to a subject an effective amount of a pharmaceutical composition comprising an antibody that binds preferentially to amino acids 28-40 of Aβ₁₋₄₀ peptide (SEQ ID NO:1) and a pharmaceutical acceptable carrier.

2. The method of claim 1, wherein the antibody binds preferentially to an epitope that includes amino acid 39 and/or 40 of the Aβ₁₋₄₀ peptide (SEQ ID NO:1).

3. The method of claim 1 or 2, wherein the antibody does not show significant cross-reactivity with Aβ₁₋₄₂ and Aβ₁₋₄₃ peptide.

4. The method of claim 1, wherein the Fab fragment of the antibody binds to Aβ₁₋₄₀ peptide (SEQ ID NO: 1) with an affinity of about 200 nM or less.

5. The method of claim 1, wherein the Fab fragment of the antibody binds Aβ₁₋₄₀ peptide (SEQ ID NO: 1) with an affinity of about 1 nM or less.

6. The method of claim 2, wherein the Fab fragment of the antibody binds Aβ₁₋₄₀ peptide (SEQ ID NO: 1) with an affinity of about 200 nM or less.

7. The method of claim 2, wherein the Fab fragment of the antibody binds Aβ₁₋₄₀ peptide (SEQ ID NO: 1) with an affinity of about 1 nM or less.

8. The method of claim 1, wherein the antibody competitively inhibits binding of a monoclonal antibody comprising amino acid sequence of SEQ ID NO: 4 and 6 to Aβ₁₋₄₀ peptide (SEQ ID NO:1).

9. The method of claim 1, wherein the antibody binds to the same epitope to which a monoclonal antibody comprising amino acid sequence of SEQ ID NO: 4 and 6 binds.

10. The method of claim 1, wherein the antibody is monoclonal antibody.

11. The method of claim 1, wherein the antibody is a humanized antibody.

12. The method of claim 1, wherein the antibody is a human antibody.

13. A method of suppressing formation of amyloid plaques in a subject comprising administering to a subject an effective amount of a pharmaceutical composition comprising an antibody that binds preferentially to amino acids 28-40 of Aβ₁₋₄₀ peptide (SEQ ID NO:1) and a pharmaceutical acceptable carrier.

14. The method of claim 13, wherein the antibody binds preferentially to an epitope that includes amino acid 39 and/or 40 of the Aβ₁₋₄₀ peptide (SEQ ID NO: 1).

15. The method of claim 13, wherein the antibody does not show significant cross-reactivity with Aβ₁₋₄₂ and Aβ₁₋₄₃ peptide.

16. The method of claim 13, wherein the Fab fragment of the antibody binds to Aβ₁₋₄₀ peptide (SEQ ID NO:1) with an affinity of about 200 nM or less.

17. The method of claim 13, wherein the Fab fragment of the antibody binds Aβ₁₋₄₀ peptide (SEQ ID NO: 1) with an affinity of about 1 nM or less.

18. The method of claim 14, wherein the Fab fragment of the antibody binds Aβ₁₋₄₀ peptide (SEQ ID NO: 1) with an affinity of about 200 nM or less.

19. The method of claim 14, wherein the Fab fragment of the antibody binds Aβ₁₋₄₀ peptide (SEQ ID NO:1) with an affinity of about 1 nM or less.

20. The method of claim 13, wherein the antibody competitively inhibits binding of a monoclonal antibody comprising amino acid sequence of SEQ ID NO: 4 and 6 to Aβ₁₋₄₀ peptide (SEQ ID NO:1).

21. The method of claim 13, wherein the antibody binds to the same epitope to which a monoclonal antibody comprising amino acid sequence of SEQ ID NO: 4 and 6 binds.

22. The method of claim 13, wherein the antibody is monoclonal antibody.

23. The method of claim 13, wherein the antibody is a humanized antibody.

24. The method of claim 13, wherein the antibody is a human antibody.

25. The method of claim 13, wherein the amyloid plaques are in the brain of the subject.

26. A method of reducing amyloid plaques in a subject comprising administering to a subject an effective amount of a pharmaceutical composition comprising an antibody that binds preferentially to amino acids 28-40 of Aβ₁₋₄₀ peptide (SEQ ID NO: 1) and a pharmaceutical acceptable carrier.

27. The method of claim 26, wherein the antibody binds preferentially to an epitope that includes amino acid 39 and/or 40 of the Aβ₁₋₄₀ peptide (SEQ ID NO:1).

28. The method of claim 26, wherein the antibody does not show significant cross-reactivity with Aβ₁₋₄₂ and Aβ₁₋₄₃ peptide.

29. The method of claim 26, wherein the Fab fragment of the antibody binds to Aβ₁₋₄₀ peptide (SEQ ID NO: 1) with an affinity of about 200 nM or less.

30. The method of claim 26, wherein the Fab fragment of the antibody binds Aβ₁₋₄₀ peptide (SEQ ID NO: 1) with an affinity of about 1 nM or less.

31. The method of claim 26, wherein the Fab fragment of the antibody binds Aβ₁₋₄₀ peptide (SEQ ID NO: 1) with an affinity of about 200 nM or less.

32. The method of claim 27, wherein the Fab fragment of the antibody binds Aβ₁₋₄₀ peptide (SEQ ID NO: 1) with an affinity of about 1 nM or less.

33. The method of claim 26, wherein the antibody competitively inhibits binding of a monoclonal antibody comprising amino acid sequence of SEQ ID NO: 4 and 6 to Aβ₁₋₄₀ peptide (SEQ ID NO:1).

34. The method of claim 26, wherein the antibody binds to the same epitope to which a monoclonal antibody comprising amino acid sequence of SEQ ID NO: 4 and 6 binds.

35. The method of claim 26, wherein the antibody is monoclonal antibody.

36. The method of claim 26, wherein the antibody is a humanized antibody.

37. The method of claim 26, wherein the antibody is a human antibody.

38. The method of claim 26, wherein the amyloid plaques are in the brain of the subject.

39. A method of delaying development of a symptom associated with Alzheimer's disease in a subject comprising administering to the subject an effective amount of a pharmaceutical composition comprising an antibody that binds preferentially to amino acids 28-40 of Aβ₁₋₄₀ peptide (SEQ ID NO:1) and a pharmaceutical acceptable carrier.

40. The method of claim 39, wherein the antibody binds preferentially to an epitope that includes amino acid 39 and/or 40 of the Aβ₁₋₄₀ peptide (SEQ ID NO:1).

41. The method of claim 39, wherein the antibody does not show significant cross- reactivity with Aβ₁₋₄₂ and Aβ₁₋₄₃ peptide.

42. The method of claim 39, wherein the Fab fragment of the antibody binds to Aβ₁₋₄₀ peptide (SEQ ID NO:1) with an affinity of about 200 nM or less.

43. The method of claim 39, wherein the Fab fragment of the antibody binds Aβ₁₋₄₀ peptide (SEQ ID NO: 1) with an affinity of about 1 nM or less.

44. The method of claim 40, wherein the Fab fragment of the antibody binds Aβ₁₋₄₀ peptide (SEQ ID NO:1) with an affinity of about 200 nM or less.

45. The method of claim 40, wherein the Fab fragment of the antibody binds Aβ₁₋₄₀ peptide (SEQ ID NO: 1) with an affinity of about 1 nM or less.

46. The method of claim 39, wherein the antibody competitively inhibits binding of a monoclonal antibody comprising amino acid sequence of SEQ ID NO: 4 and 6 to Aβ₁₋₄₀ peptide (SEQ ID NO:1).

47. The method of claim 39, wherein the antibody binds to the same epitope to which a monoclonal antibody comprising amino acid sequence of SEQ ID NO: 4 and 6 binds.

48. The method of claim 39, wherein the antibody is monoclonal antibody.

49. The method of claim 39, wherein the antibody is a humanized antibody.

50. The method of claim 39, wherein the antibody is a human antibody.
